(19) United States
(12) Patent Application Publication
(10) Pub. No.: US 2010/0273226 A1
(43) Pub. Date: Oct. 28, 2010

(54) ISOLATED POLYNUCLEOTIDE FOR INCREASING ALCOHOL TOLERANCE OF HOST CELL, VECTOR AND HOST CELL CONTAINING THE SAME, AND METHOD OF PRODUCING ALCOHOL USING THE SAME

(75) Inventors: Byung Jo YU, Hwaseong-si (KR); Jae Chan PARK, Yongin-si (KR); Sung Min PARK, Yongin-si (KR); Dae Hyeok KWEON, Suwon-si (KR); Min Eui HONG, Suwon-si (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 12/558,412

(22) Filed: Sep. 11, 2009

(30) Foreign Application Priority Data
Apr. 24, 2009 (KR) 10-2009-0036253

(51) Int. Cl. C12P 7/02; C07H 21/00; C07H 21/04; C12N 15/74; C12N 1/19

(52) U.S. Cl. 435/155; 536/23.1; 536/23.74; 435/320.1; 435/254.21

(57) ABSTRACT

Provided herein is an isolated polynucleotide for increasing the alcohol tolerance of a host cell. Also disclosed herein are a vector and a host cell containing the isolated polynucleotide, and a method of increasing the volumetric productivity of a bioalcohol using the same.

### CROSS-REFERENCE TO RELATED APPLICATION

This application claims priority to Korean Patent Application. No. 10-2009-0036253, filed on Apr. 24, 2009, and all the benefits accruing therefrom under, 35 U.S.C. §119, the contents of which in its entirety is herein incorporated by reference.

### BACKGROUND

1. Field

Exemplary embodiments relate to an isolated polynucleotide for increasing the alcohol tolerance of a host cell, a vector and a host cell containing the polynucleotide, and a method of producing alcohol using the same.

2. Description of the Related Art

With globally increasing concern about the exhaustion of resources and pollution of the environment by overuse of fossil fuels, the development of novel and renewable alternative energy sources that stably and continuously produce energy is being considered. As an example of this development of alternative energy, the technology for producing bio alcohol from biomass has been attracting considerable attention.

Today, first generation biofuels using saccharides such as a sugar cane, or starches such as a corn, are being produced. In addition, second generation biofuels are being developed using wood sources, specifically lignocelluloses, which are considered the most abundant, rich and renewable sources in the world. In recent times, the development of biofuels using algae has also been progressing.

Processes of producing these biofuels include pretreating biomass to facilitate saccharification, saccharifying the pretreated biomass to convert the pretreated biomass into monosaccharides, and fermenting the monosaccharides to produce bioalcohol.

The fermentation process involves the biological oxidation of an organic compound utilizing fermentation bacteria such as yeast, etc. Bacterial metabolism occurs through various different mechanisms depending on the bacterial species and environmental conditions used. All heterotropic bacteria generate energy through the oxidation of organic compounds such as carbohydrates (e.g., glucoses), lipids, and proteins.

The general process by which bacteria metabolize suitable substrates is glycolysis. Glycolysis is a sequence of reactions that converts glucose into pyruvate in order to generate ATP. In production of metabolic energy, the fate of pyruvate varies depending on the bacterial species and environmental conditions.

There are three principle reactions of pyruvate. First, under aerobic conditions, many microorganisms will produce energy via the citric acid cycle and the conversion of pyruvate into acetyl coenzyme A, catalysed by the enzyme pyruvate dehydrogenase (PDH).

Second, under anaerobic conditions, certain ethanologenic organisms can carry out alcoholic fermentation by the decarboxylation of pyruvate into acetaldehyde, catalysed by the enzyme pyruvate decarboxylase (PDC), and the subsequent reduction of acetaldehyde into ethanol by nicotinamide adenine dinucleotide (NADH), catalysed by the enzyme alcohol dehydrogenase (ADH).

Third, pyruvate is converted into lactate through catalysis by the enzyme lactate dehydrogenase (LDH).

There has been much interest in producing ethanol using either microorganisms that undergo anaerobic fermentation naturally, or through the use of host cells which incorporate the pyruvate decarboxylase and alcohol dehydrogenase genes.

However, since microorganisms generally have a low alcohol tolerance, the microorganisms may be damaged by alcohol that is produced by the microorganisms if the alcohol concentration becomes too high, and may die if the alcohol concentration exceeds 15%.

For this reason, research into improving the volumetric productivity of alcohol through an optimized fermentation process and through improved strains of microorganisms, has been conducted by industries associated with alcohol fermentation and production in order to obtain economical advantages.

In recent times, the technology for increasing alcohol tolerance using spt-modified strains of yeast has been developed.

However, the discovery of various gene groups involved in ethanol tolerance and the discovery of a variety of novel gene groups further involved in alcohol tolerance are needed for the production of a second generation energy such as isobutanol.

### SUMMARY

Exemplary embodiments provide strains that exhibit high viability and homeostasis by increasing the alcohol tolerance of a microorganism, and thus are widely applied to alcohol fermentation processes through various genetic disturbances. Other exemplary embodiments provide a method of producing bioalcohol with high volumetric productivity using strains having excellent fermentation ability and excellent fermentation maintaining ability.

In one embodiment, an isolated polynucleotide encoding a polypeptide for increasing alcohol tolerance and/or the volumetric productivity of alcohol of a host cell is provided.

In one embodiment, the isolated polynucleotide is at least one polynucleotide selected from the group consisting of: a polynucleotide consisting of a base sequence having at least 90% identity to a base sequence selected from SEQ ID NOs: 1 to 8; a polynucleotide encoding a polypeptide consisting of an amino acid sequence having at least 90% identity to an amino acid sequence selected from SEQ ID NOs: 14 to 19; a polynucleotide consisting of a base sequence which hybridizes to a base sequence selected from SEQ ID NOs: 1 to 8 under stringent conditions; and a polynucleotide encoding a polypeptide consisting of an amino acid sequence which hybridizes to an amino acid sequence selected from SEQ ID NOs: 14 to 19 under stringent conditions.

In another embodiment, a vector containing the isolated polynucleotide is provided.

In yet another embodiment, a host cell capable of producing alcohol when incubated in a monosaccharide-containing nutrient source is provided. In yet a further embodiment, the host cell encodes for a polypeptide which increases the alcohol tolerance of the host cell.

In one embodiment, the host cell exhibits overexpression of one or more isolated polynucleotides encoding a polypeptide for increasing alcohol tolerance of the host cell, wherein the isolated polynucleotide is selected from the group consisting of: a polynucleotide consisting of a base sequence having at least 90% identity to a base sequence selected from SEQ ID NOs: 1 to 8; a polynucleotide encoding a polypeptide consisting of an amino acid sequence having at least 90% identity to an amino acid sequence selected from SEQ ID NOs: 14 to 19; a polynucleotide consisting of a base sequence which hybridizes to a base sequence selected from SEQ ID NOs: 1 to 8 under stringent conditions; and a polynucleotide encoding a polypeptide consisting of an amino acid sequence which hybridizes to an amino acid sequence selected from SEQ ID NOs: 14 to 19 under stringent conditions.

In another embodiment, a method of producing bioalcohol using the host cell is provided. The method includes a fermentation process including incubating a host cell in a monosaccharide-containing nutrient media and producing bioalcohol.

In yet another embodiment, the method of producing bioalcohol is performed by engineering a host cell to overexpress one or more isolated polynucleotides encoding a polypeptide for increasing the alcohol tolerance of the host cell, selected from the group consisting of a polynucleotide consisting of a base sequence having at least 90% identity to a base sequence selected from SEQ ID NOs: 1 to 8, a polynucleotide encoding a polypeptide consisting of an amino acid sequence having at least 90% identity to an amino acid sequence selected from SEQ ID NOs: 14 to 19, a polynucleotide consisting of a base sequence hybridized to a base sequence selected from SEQ ID NOs: 1 to 8 under stringent conditions, and a polynucleotide encoding a polypeptide consisting of an amino acid sequence hybridized to an amino acid sequence selected from SEQ ID NOs: 14 to 19 under stringent conditions; and incubating the host cell in a monosaccharide-containing nutrient source under suitable conditions for a predetermined period of time to produce alcohol through fermentation.

### BRIEF DESCRIPTION OF THE DRAWINGS

The above and other aspects, advantages and features of this disclosure will become more apparent by describing in further detail exemplary embodiments thereof with reference to the accompanying drawings, in which:

FIG. 1 is a map of an open reading frame (ORF) of a 1^{st} polynucleotide according to an exemplary embodiment;

FIG. 2 is a map of an ORF of a 2^{nd} polynucleotide according to an exemplary embodiment;

FIG. 3 is a map of an ORF of a 3^{rd} polynucleotide according to an exemplary embodiment;

FIG. 4 is a map of an ORF of a 4^{th} polynucleotide according to an exemplary embodiment;

FIG. 5 is a map of an ORF of a 5^{th} polynucleotide according to an exemplary embodiment;

FIG. 6 is a map of an ORF of a 6^{th} polynucleotide according to an exemplary embodiment;

FIG. 7 is a map of an ORF of a 7^{th} polynucleotide according to an exemplary embodiment;

FIG. 8 is a map of an ORF of an 8^{th} polynucleotide according to an exemplary embodiment;

FIG. 9 is a graph showing the results of a 5% (w/v) ethanol tolerance test performed in a liquid medium according to Experimental Example 1;

FIG. 10 is a graph illustrating the results of a 1% (w/v) isobutanol tolerance test performed in a liquid medium according to Experimental Example 2;

FIG. 11 is a graph illustrating the volumetric production of ethanol in a liquid medium according to Experimental Example 1;

FIG. 12 shows the results of an ethanol tolerance test performed in a solid medium according to Experimental Example 3;

FIG. 13 shows the results of an isobutanol tolerance test performed in a solid medium according to Experimental Example 4;

FIG. 14 is a graph illustrating the results of fermentation tests in 5% ethanol and 10% for the bacterial strains of Experimental Example 1;

FIG. 15 is a graph illustrating the results of fermentation tests in 5% ethanol and 10% for the bacterial strains of Experimental Example 2;

FIG. 16 is a graph illustrating the results of fermentation tests in 5% ethanol and 10% for the bacterial strains of Experimental Example 3;

FIG. 17 is a graph illustrating the results of fermentation tests in 5% ethanol and 10% for the bacterial strains of Experimental Example 4;

FIG. 18 is a graph illustrating the results of fermentation tests in 5% ethanol and 20% glucose for the bacterial strains of Experimental Example 1;

FIG. 19 is a graph illustrating the results of fermentation tests in 5% ethanol and 20% glucose for the bacterial strains of Experimental Example 2;

FIG. 20 is a graph illustrating the results of fermentation tests in 5% ethanol and 20% glucose for the bacterial strains of Experimental Example 3;

FIG. 21 is a graph illustrating the results of fermentation tests in 5% ethanol and 20% glucose for the bacterial strains of Experimental Example 4;

FIG. 22 is a graph illustrating the results of fermentation tests in 10% (w/v) glucose using a low cell inoculum according to Experimental Example 7;

FIG. 23 is a graph illustrating the results of fermentation tests in 10% (w/v) glucose using a high cell inoculum according to Experimental Example 7;

FIG. 24 is a graph illustrating the results of fermentation tests in 20% (w/v) glucose using a low cell inoculum according to Experimental Example 7;

FIG. 25 is a graph illustrating the results of fermentation tests in 20% (w/v) glucose using a high cell inoculum according to Experimental Example 7;

FIG. 26 is a graph illustrating the results of fermentation tests in 30% (w/v) glucose using a low cell inoculum according to Experimental Example 7;

FIG. 27 is a graph illustrating the results of fermentation tests in 30% (w/v) glucose using a high cell inoculum according to Experimental Example 7;

FIG. 28 is a graph illustrating the results of fermentation tests in 2% (w/v) glucose/2% (w/v) galactose according to Experimental Example 8;

FIG. 29 is a graph illustrating the results of fermentation tests in 2% (w/v) glucose/6% (w/v) galactose according to Experimental Example 8;

FIG. 30 is a graph illustrating the results of fermentation tests in 2% (w/v) glucose/8% (w/v) galactose according to Experimental Example 8;

FIG. 31 is a graph illustrating the relative viable cell count versus time for ethanol tolerance tests performed in 15% (w/v) ethanol media, based on colony forming units (CFUs), according to Experimental Example 9; and

FIG. 32 is a graph illustrating the Mean of LN(relative viable cell count) versus time (cell death rate) for ethanol tolerance tests preformed in 15% (w/v) ethanol media, based on CFUs, according to Experimental Example 9.

### DETAILED DESCRIPTION

Hereinafter, advantages, features and methods for embodying the inventive concept will be described more fully with reference to the detailed descriptions of the following exemplary embodiments and the accompanying drawings. However, the inventive concept is not limited to the described example embodiments, and thus may be embodied in various forms.

In addition, it would be understood that all the numbers representing contents and conditions used in the specification and claims may be changed. Thus, unless indicated otherwise, a numeral parameter shown in the specification and accompanying claims is an approximation that may be changed according to the purpose of the inventive concept.

The terminology used herein is for the purpose of describing particular embodiments only and is not intended to be limiting of the invention. The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e. meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

1. Isolated Polynucleotide

According to an exemplary embodiment, an isolated polynucleotide encoding a protein that increases alcohol tolerance is provided.

In one exemplary embodiment, the isolated polynucleotide may include a polynucleotide consisting of a base sequence selected from any one of SEQ ID NOs: 1 to 8, which encode a protein that increases alcohol tolerance. Alternatively, the isolated polynucleotide may include a polynucleotide with a base sequence having at least about 70, about 75, about 80, about 85, about 90, about 95 or about 99% identity to the above-mentioned base sequences and which has the above-mentioned activity. The isolated polynucleotide may be a fragment or variant of the polynucleotide, or a polynucleotide that hybridizes to the polynucleotide under stringent conditions.

In another exemplary embodiment, the isolated polynucleotide may be a polynucleotide encoding a polypeptide consisting of an amino acid sequence selected from SEQ ID NOs: 14 to 19 and that increases the alcohol tolerance of a host cell. Alternatively, the isolated polynucleotide may include a polynucleotide encoding a polypeptide having at least about 70, about 75, about 80, about 85, about 90, about 95 or about 99% identity to the above-mentioned amino acid sequence and having the above-mentioned activity, or may include a fragment or variant of the polynucleotide, or a polynucleotide encoding a polypeptide consisting of an amino acid sequence that hybridizes to the above-mentioned polypeptide under stringent conditions.

In an exemplary embodiment, the isolated polynucleotide may be selected from the following:

(a) a polynucleotide consisting of a base sequence having at least 90% identity to a base sequence selected from SEQ ID NOs: 1 to 8;

(b) a polynucleotide encoding a polypeptide consisting of an amino acid sequence having at least 90% identity to an amino acid sequence selected from SEQ ID NOs: 14 to 19;

(c) a polynucleotide consisting of a base sequence which hybridizes to a base sequence selected from SEQ ID NOs: 1 to 8 under stringent conditions; and

(d) a polynucleotide encoding a polypeptide consisting of an amino acid sequence which hybridizes to an amino acid sequence selected from SEQ ID NOs: 14 to 19 under stringent conditions.

The isolated polynucleotide may be derived from yeast, for example, Saccharomyces cerevisiae.

Since the isolated polynucleotide encodes a protein that increases alcohol tolerance, a host cell containing the same exhibits excellent viability in the presence of high concentrations of alcohol and excellent homeostasis during fermentation. Thus, when it is used in industrial alcohol fermentation, alcohol volumetric productivity may be increased.

The technical and scientific terms used herein have meanings conventionally understood by those skilled in the art unless there are specific descriptions. The terms as used herein have the following meanings.

The term “polynucleotide” generally refers to a non-modified or modified polyribonucleotide (e.g. RNA) or polydeoxyribonucleotide (e.g. DNA). Examples of the “polynucleotide” include, but are not limited to, single- or double-stranded DNA; DNA that is a mixture of single- and double-stranded regions; single- or double-stranded RNA; RNA that is a mixture of single- and double-stranded regions; hybrid molecules including single- or double stranded DNA or RNA; or DNA or RNA that is a mixture of single- and double-stranded regions. In addition, the “polynucleotide” may include a triple-stranded region having RNA or DNA, or both RNA and DNA, or may include a relatively short polynucleotide, often referred to as an oligonucleotide.

The term “isolated”, when used to describe the various polynucleotides or polypeptides, means a polynucleotide or polypeptide that has been identified and separated and/or recovered from a component of its natural environment. For example, a polynucleotide or polypeptide present in the original living organism is not “isolated,” but the same polynucleotide or polypeptide removed from the natural co-existing material is “isolated.” The term also embraces recombinant polynucleotides and polypeptides and chemically synthesized polynucleotides and polypeptides. Further, a polynucleotide or polypeptide introduced into a living organism by transformation, genetic engineering or by other recombination techniques is considered “isolated” even though it is present in a living organism.

The term “polypeptide” refers to a peptide or protein containing two or more amino acids linked to each other by peptide bonds or by modified peptide bonds. The “polypeptide” includes short chains such as peptides, oligopeptides or oligomers, and to long chains such as proteins. The “polypeptide” may include amino acids other than the 20 gene-encoded amino acids. The “polypeptide” includes amino acid sequences modified by natural processes or by chemical modification techniques known in the art. The modifications to the “polypeptide” include acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, crosslinking, cyclization, disulfide bond formation, demethylation, formation of covalent crosslinks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

The term “fragment” of a polynucleotide sequence refers to a polynucleotide sequence that is shorter than the reference sequence in the sequence listing. The “fragment” of the polypeptide sequence is a polypeptide sequence that is shorter than the reference sequence, but which has substantially the same biological function or activity as the reference polypeptide.

The term “variant” refers to a polynucleotide or polypeptide that differs from, but has the same basic properties as, a reference polynucleotide or polypeptide. A typical variant of a polynucleotide differs in nucleotide sequence from the reference polynucleotide. Changes in the nucleotide sequence of the variant may or may not alter the amino acid sequence of a polypeptide encoded by the reference polynucleotide. The nucleotide changes may result in amino acid substitutions, additions, deletions, fusions and/or truncations in the polypeptide encoded by the reference sequence. A typical variant of a polypeptide differs in amino acid sequence from the reference polypeptide. Generally, the alterations are limited so that the sequences of the reference polypeptide and the variant are closely similar overall and, in many regions, are identical. A variant polypeptide and a reference polypeptide may differ in amino acid sequence by one or more substitutions, insertions, or deletions in any combination. A substituted or inserted amino acid residue may or may not be one encoded by a genetic code. Typical conservative substitutions include Gly, Ala; Val, Ile, Leu; Asp, Glu; Asn, Gm; Ser, Thr; Lys, Arg; and Phe and Tyr. A variant of a polynucleotide or polypeptide may be naturally occurring such as within an allele, or it may be a variant that is not known to occur naturally. Non-naturally occurring variants of polynucleotides and/or polypeptides may be made by mutagenesis techniques or by direct synthesis. A variant of a polypeptide may be a polypeptide having one or more post-translational modifications such as glycosylation, phosphorylation, methylation and ADP ribosylation, The variant of the polynucleotide may include a splice variant, an allelic variant or a polynucleotide having a single nucleotide polymorphism (SNP).

The term “stringent conditions” refers to conditions under which overnight incubation is conducted for a period of about 2.5 hours in a solution containing 6× standard sodium citrate (SSC) and 0.1% sodium dodecyl sulphate (SDS) at a temperature of 42° C., and then washing the filter in 1.0×SSC/0.1% SDS at a temperature of 65° C.

The term “identity” reflects a relationship between two or more polypeptide or polynucleotide sequences, and is determined by comparing the sequences to one another. Generally, the term “identity” refers to an exact nucleotide to nucleotide or amino acid to amino acid correspondence between the two or more polynucleotide sequences, or the two or more polypeptide sequences, respectively, over the length of the sequences being compared. Methods of comparing identity and similarity of two sequences are known in the art. For example, the percent (%) identity between two polynucleotides, and the % identity and % similarity between two polypeptide sequences, may be determined using the Wisconsin Sequence Analysis Package, version 9.1 (Devereux J. et al., Nucleic Acids Res, 12, 387-395 (1984); available from Genetics Computer Group, Madison Wis., USA); such as the programs BESTFIT and GAP.

The program BESTFIT finds the best single region of similarity between two sequences using the “local homology” algorithm of Smith and Waterman (Advances in Applied Mathematics, 2:482-489, 1981). BESTFIT is more suitable for comparing two polynucleotide sequences or two polypeptide sequences that are not similar in length, and assumes that the shorter sequence is representative of a longer portion.

In comparison, the program GAP aligns two sequences to find a “maximum similarity”, according to the Needleman-Wunsch algorithm (J. Mol. Biol. 48:443-354, 1970). GAP is more suitable for comparing sequences having approximately the same length, and expects that alignment will be made over the entire length. The parameters of “gap weight” and “length weight” used in each program are 50 and 3 for polynucleotide sequences, and 12 and 4 for polypeptide sequences, respectively.

Other programs for determining identity and/or similarity between sequences include the BLAST family of programs (Altschul S. F. et al., Nucleic Acids Res., 25:389-3402 (1997), available from the National Center for Biotechnology Information (NCBI), and FASTA (Pearson W. R., Methods in Enzymology, 183, 63-99 (1990)).

The terms “increase in alcohol tolerance” or “increase in alcohol resistance” may be used interchangeably and mean an improvement in the resistance of a host cell to alcohol. The increase in alcohol tolerance may be observed by comparing the cell growth rate of the wild-type cell and the control cell (transformed with an empty vector) and determining the minimum inhibitory concentration (MIC), the final cell density, and decreased lag time.

The polynucleotide consisting of a base sequence selected from SEQ ID NOs: 1 to 8, or the polynucleotide encoding a polypeptide consisting of an amino acid sequence selected from SEQ ID NOs: 14 to 19, includes all or partial genes listed in Table 1 below.

TABLE 1

| No. of | No. of | | |
|---|---|---|---|
| Base | Amino Acid | | |
| Sequence | Sequence | Gene | Name |
| 1 | 14 | truncated MIH1 | 1^{st} polynucleotide |
| 2 | 15 | INO1 | 2^{nd} polynucleotide |
| 3 | 16 | DOG1 | 3^{rd} polynucleotide |
| 4 | 17 | HAL1 | 4^{th} polynucleotide |
| 5 | 18 | TRP1 | 5^{th} polynucleotide |
| 6 | 19 | truncated MRPL17 | 6^{th} polynucleotide |
| 7 | — | Partial fragment | 7^{th} polynucleotide |
| | | YLR157C-B | |
| 8 | — | putative SPG5 promoter | 8^{th} polynucleotide |

Hereinafter, each polynucleotide will be described in detail.

In one exemplary embodiment, the isolated polynucleotide encoding a polypeptide which increases the alcohol tolerance of a host cell may include a first polynucleotide

(hereinafter, referred to as a “1^{st} polynucleotide”) selected from the following:

(i) a polynucleotide consisting of a base sequence having at least 90% identity to a base sequence of SEQ ID NO: 1;

(ii) a polynucleotide encoding a polypeptide consisting of an amino acid sequence having at least 90% identity to an amino acid sequence of SEQ ID NO: 14;

(iii) a polynucleotide consisting of a base sequence which hybridizes to a base sequence of SEQ ID NO: 1 under stringent conditions; and

(iv) a polynucleotide encoding a polypeptide consisting of an amino acid sequence which hybridizes to an amino acid sequence of SEQ ID NO: 14 under stringent conditions.

In the 1^{st} polynucleotide, the base sequence set forth in SEQ ID NO: 1, or the amino acid sequence set forth in SEQ ID NO: 14, encodes for a partial MIH1 gene. That is, the 1^{st} polynucleotide encodes for a partial MIH1 gene (hereinafter, referred to as “truncated MIH1”) from which the 126^{th} to 555^{th} amino acids are deleted, resulting in a polypeptide which encodes for the 1^{st} to 125^{th} amino acids.

The MIH1 gene is a cell cycle regulator, which serves as a tyrosine phosphatase involved in the extension of the G2 phase during the Yeast cell cycle. However, since the 126^{th} to 555^{th} amino acids are deleted, it is estimated that the truncated MIH1 gene will not be able to control the cell cycle, resulting in continuous cell growth.

The 1^{st} polynucleotide may be an isolated polynucleotide selected from the following:

(i) an isolated polynucleotide consisting of base sequences having at least 90% identity to base sequences of SEQ ID NOs: 1 and 9;

(ii) an isolated polynucleotide encoding a polypeptide consisting of amino acid sequences having at least 90% identity to amino acid sequences of SEQ ID NOs: 14 and 21;

(iii) an isolated polynucleotide which hybridizes to the isolated polynucleotide of (i) under stringent conditions; and

(iv) an isolated polynucleotide which hybridizes to the isolated polynucleotide of (ii) under stringent conditions.

The isolated polynucleotide of (i) may be an isolated polynucleotide (hereinafter, referred to as a “1-1^{st} polynucleotide”) having a base sequence set forth in SEQ ID NO: 26.

The 1-1^{st} polynucleotide has the genetic map shown in FIG. 1. Referring to FIG. 1, the 1-1^{st} polynucleotide is derived from the 13^{th} chromosome of S. cerevisiae, which includes a partial MSN2 gene and a partial MIH1 gene.

In the 1-1^{st} polynucleotide, the nucleotide base sequence set forth in SEQ ID NO: 9, or the amino acid sequence set forth in SEQ ID NO: 21, encode for a partial MSN2 gene.

The MSN2 gene encodes for a transcription factor expressed by cells in response to various stresses received from an external environment. The product of the expressed MSN2 gene binds to the promoter region of various gene groups in a nucleus having stress response elements (“STREs”), which are specific recognition sites, expressing the STREs.

However, the 1-1^{st} polynucleotide encodes for a partial MSN2 gene (hereinafter referred to as “truncated MSN2”) from which the 1^{st} to 48^{th} amino acids are deleted, resulting in a gene encoding for only 656 amino acids (49^{th} to 705^{th} amino acids). The deleted site is an activation domain site that binds to and thus activates the YAK1 gene, thereby stopping cell growth. When the truncated MSN2 gene is expressed, it is anticipated that it will compete with the intact MSN2 gene product and thus inhibit activity of the YAK1 gene, resulting in cell growth under stress conditions (e.g. a high concentration of ethanol).

In another exemplary embodiment, the isolated polynucleotide encoding a polypeptide increasing alcohol tolerance of the host cell may include a second polynucleotide (hereinafter, referred to as a “2^{nd} polynucleotide”) selected from the following:

(i) a polynucleotide consisting of a base sequence having at least 90% identity to a base sequence of SEQ ID NO: 2;

(ii) a polynucleotide encoding a polypeptide consisting of an amino acid sequence having at least 90% identity to an amino acid sequence of SEQ ID NO: 15;

(iii) a polynucleotide consisting of a base sequence which hybridizes to a base sequence of SEQ ID NO: 2 under stringent conditions; and

(iv) a polynucleotide encoding a polypeptide consisting of an amino acid sequence which hybridizes to an amino acid sequence of SEQ ID NO: 15 under stringent conditions.

In the 2^{nd} polynucleotide, the base sequence set forth in SEQ ID NO: 2, or the amino acid sequence set forth in SEQ ID NO: 15, encodes the IN01 gene.

The IN01 gene is a gene encoding for an inositol-1-phosphate synthase involved in the syntheses of inositol phosphate and inositol-containing phospholipid. Inositol is an essential material for the growth of a microorganism, stimulating development of the microorganism. When the IN01 gene is deleted, ethanol tolerance is rapidly decreased, and conversely, when inositol levels are excessive, ethanol tolerance is increased.

In an embodiment, the 2^{nd} polynucleotide may be an isolated polynucleotide selected from the following:

(i) an isolated polynucleotide consisting of base sequences having at least 90% identity to base sequences of SEQ ID NOs: 2, 10 and 11;

(ii) an isolated polynucleotide encoding a polypeptide consisting of amino acid sequences having at least 90% identity to amino acid sequences of SEQ ID NOs: 15, 22 and 23;

(iii) an isolated polynucleotide which hybridizes to the isolated polynucleotide of (i) under stringent conditions; and

(iv) an isolated polynucleotide which hybridizes to the isolated polynucleotide of (ii) under stringent conditions.

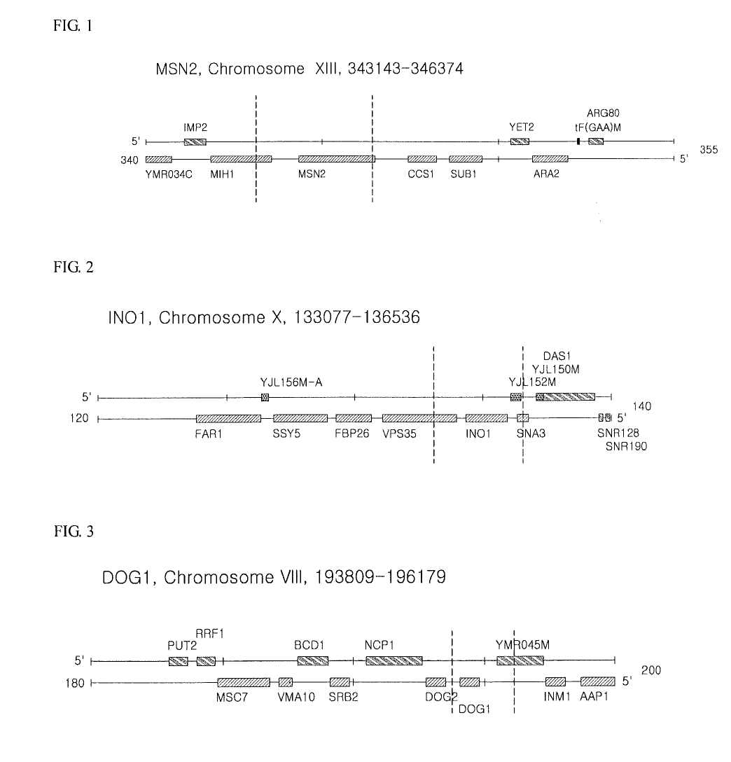

The isolated polynucleotide of (i) may be an isolated polynucleotide (hereinafter, referred to as a “2-1^{st} polynucleotide”) having a base sequence set forth in SEQ ID NO: 27 and having the genetic map as shown in FIG. 2.

Referring to FIG. 2, the 2-1^{st} polynucleotide is derived from the 10^{th} chromosome of S. cerevisiae, which encodes the IN01 gene as described above.

Further, in the 2-1^{st} polynucleotide, the base sequence set forth in SEQ ID NO: 10, or the amino acid sequence set forth in SEQ ID NO: 22, encodes for a partial VPS35 gene (hereinafter, referred to as “truncated VPS35”), from which the 285^{th} to 945^{th} amino acids are deleted, resulting in a VPS35 gene encoding only the 1^{st} to 284^{th} amino acids. The VPS35 gene serves to transport foreign proteins.

Furthermore, in the 2-1^{st} polynucleotide, the base sequence set forth in SEQ ID NO: 11, or the amino acid sequence set forth in SEQ ID NO: 23, encodes for a partial SNA3 gene (referred to as “truncated SNA3”) from which the 1^{st} to 77^{th} amino acids are deleted, thereby encoding only the 78^{th} to 134^{th} amino acids. The function of the SNA3 gene is not known.

In another exemplary embodiment, the isolated polynucleotide may include a third polynucleotide (referred to as a “3^{rd} polynucleotide”) selected from the following:

(i) a polynucleotide consisting of a base sequence having at least 90% identity to a base sequence of SEQ ID NO: 3;

(ii) a polynucleotide encoding a polypeptide consisting of an amino acid sequence having at least 90% identity to an amino acid sequence of SEQ ID NO: 16;

(iii) a polynucleotide consisting of a base sequence which hybridizes to a base sequence of SEQ ID NO: 3 under stringent conditions; and

(iv) a polynucleotide encoding a polypeptide consisting of an amino acid sequence which hybridizes to an amino acid sequence of SEQ ID NO: 16 under stringent conditions.

In the 3^{rd} polynucleotide, the base sequence set forth in SEQ ID NO: 3, or the amino acid sequence set forth in SEQ ID NO: 16, encodes the DOG1 gene.

The DOG1 gene encodes for a 2-deoxyglucose-6-phophatase and confers tolerance to 2-deoxyglucose when when 2-deoxyglucose is overexpressed.

The 3^{rd} polynucleotide may be an isolated polynucleotide selected from the following:

(i) an isolated polynucleotide consisting of base sequences having at least 90% identity to base sequences of SEQ ID NOs: 3 and 12;

(ii) an isolated polynucleotide consisting of amino acid sequences having at least 90% identity to amino acid sequences of SEQ ID NOs: 16 and 24;

(iii) an isolated polynucleotide which hybridizes to the isolated polynucleotide of (i) under stringent conditions; and

(iv) an isolated polynucleotide which hybridizes to the isolated polynucleotide of (ii) under stringent conditions.

The isolated polynucleotide of (i) may be a polynucleotide (referred to as a “3-1^{st} polynucleotide”) having a base sequence set forth in SEQ ID NO: 28 and having the genetic map as shown in FIG. 3.

Referring to FIG. 3, the 3-1^{st} polynucleotide is derived from the 8^{th} chromosome of S. cerevisiae, and encodes the DOG1 gene as described above. In the 3-1^{st} polynucleotide, the base sequence set forth in SEQ ID NO: 12, or the amino acid sequence set forth in SEQ ID NO: 24, encodes a partial YHRO45W gene (referred to as “truncated YHRO45W”) from which the 213^{th} to 561^{st} amino acids are deleted, thereby encoding for only the 1^{st} to 212^{th} amino acids.

While the function of the YHRO45W gene is not known, the YHRO45W gene is known to encode for a green fluorescent protein (GFP)-fusion protein located in a vesicle (Huh W K, et al. (2003) Global analysis of protein localization in budding yeast, Nature 425(6959):686-91).

In one exemplary embodiment, the isolated polynucleotide may include a fourth polynucleotide (referred to as a “4^{th} polynucleotide”) selected from the following:

(i) a polynucleotide consisting of a base sequence having at least 90% identity to a base sequence of SEQ ID NO: 4;

(ii) a polynucleotide encoding a polypeptide consisting of an amino acid sequence having at least 90% identity to an amino acid sequence of SEQ ID NO: 17;

(iii) a polynucleotide consisting of a base sequence which hybridizes to a base sequence of SEQ ID NO: 4 under stringent conditions; and

(iv) a polynucleotide encoding a polypeptide consisting of an amino acid sequence which hybridizes to an amino acid sequence of SEQ ID NO: 17 under stringent conditions.

In the 4^{th} polynucleotide, the polynucleotide consisting of the base sequence set forth in SEQ ID NO: 4, or encoding a polypeptide consisting of the amino acid sequence set forth in SEQ ID NO: 17, encodes the HAL1 gene.

The HAL1 gene encodes for a cytoplasmic protein involved in halotolerance. Expression of the HAL1 gene is inhibited by Ssn6p-Tup1p and Sko1p, and is induced by NaCl, KCl and sorbitol via Gcn4p (refer to Marquez J. A., et al. (1998) The Ssn6-Tup1 repressor complex of Saccharomyces cerevisiae is involved in the osmotic induction of HOG-dependent and -independent genes EMBO J. 17(9):2543-53; and Pascual-Ahuir A, et al. (2001) The Sko1p repressor and Gcn4p activator antagonistically modulate stress-regulated transcription in Saccharomyces cerevisiae. Mol Cell Biol 21(1):16-25)

The 4^{th} polynucleotide may be an isolated polynucleotide selected from the following:

(i) an isolated polynucleotide consisting of base sequences having at least 90% identity to base sequences of SEQ ID NOs: 4 and 13;

(ii) an isolated polynucleotide consisting of amino acid sequences having at least 90% identity to amino acid sequences of SEQ ID NOs: 17 and 25;

(iii) an isolated polynucleotide which hybridizes to the isolated polynucleotide of (i) under stringent conditions; and

(iv) an isolated polynucleotide which hybridizes to the isolated polynucleotide of (ii) under stringent conditions.

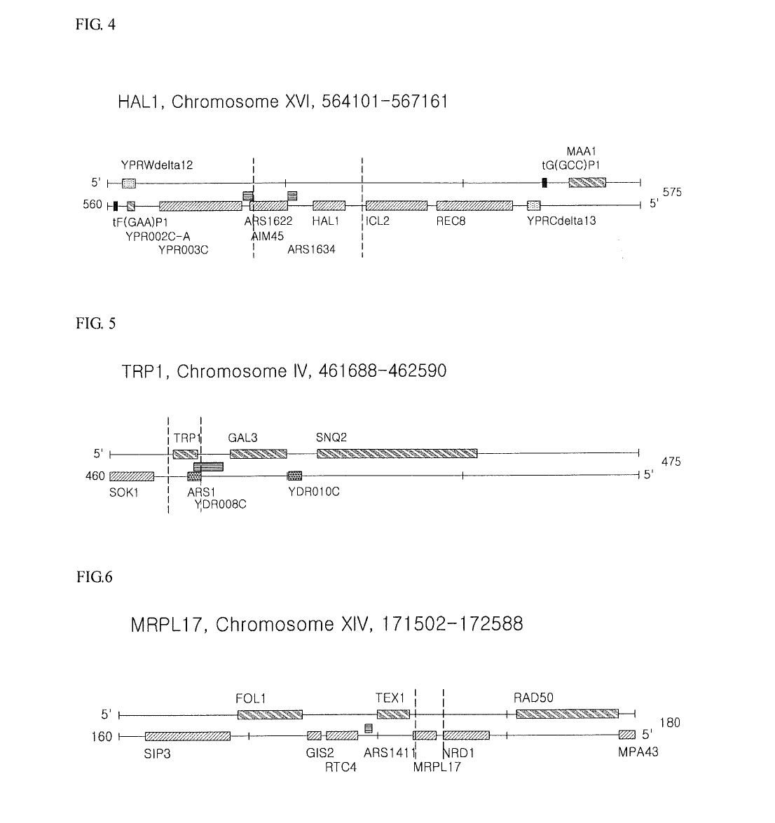

The isolated polynucleotide of (i) may be an isolated polynucleotide (referred to as a “4-1^{st} polynucleotide”) having the base sequence set forth in SEQ ID NO: 29 and having a genetic map as shown in FIG. 4.

Referring to FIG. 4, the 4-1^{st} polynucleotide, derived from the 16^{th} chromosome of S. cerevisiae, encodes the HAL1 gene as described above. In the 4-1^{st} polynucleotide, the base sequence set forth in SEQ ID NO: 13 or the amino acid sequence set forth in SEQ ID NO: 25 encodes for a partial AIM45 gene (referred to as “truncated AIM45”) from which the 313^{th} to 345^{th} amino acids are deleted, thereby encoding only the 1^{st} to 312^{th} amino acids. The function of the AIM45 gene is not known.

In one exemplary embodiment, the isolated polynucleotide may include a fifth polynucleotide (referred to as a “5^{th} polynucleotide”) selected from the following:

(i) a polynucleotide consisting of a base sequence having at least 90% identity to a base sequence of SEQ ID NO: 5;

(ii) a polynucleotide encoding a polypeptide consisting of an amino acid sequence having at least 90% identity to an amino acid sequence of SEQ ID NO: 18;

(iii) a polynucleotide consisting of a base sequence which hybridizes to a base sequence of SEQ ID NO: 5 under stringent conditions; and

(iv) a polynucleotide encoding a polypeptide consisting of an amino acid sequence which hybridizes to an amino acid sequence of SEQ ID NO: 18 under stringent conditions.

In the 5^{th} polynucleotide, the base sequence set forth in SEQ ID NO: 5, or the amino acid sequence set forth in SEQ ID NO: 18, encodes the TRP1 gene.

The TRP1 gene encodes for a phosphoribosylanthranilate isomerase which catalyzes the third step of tryptophan biosynthesis.

The 5^{th} polynucleotide may be an isolated polynucleotide (referred to as a “5-1^{st} polynucleotide”) having the base sequence set forth in SEQ ID NO: 30 and having a genetic map as shown in FIG. 5.

Referring to FIG. 5, the 5-1^{st} polynucleotide, derived from the 4^{th} chromosome of S. cerevisiae, encodes the TRP1 gene as described above.

In another exemplary embodiment, the isolated polynucleotide may include a sixth polynucleotide (referred to as a “6^{th} polynucleotide”) selected from the following:

(i) a polynucleotide consisting of a base sequence having at least 90% identity to a base sequence of SEQ ID NO: 6;

(ii) a polynucleotide encoding a polypeptide consisting of an amino acid sequence having at least 90% identity to an amino acid sequence of SEQ ID NO 19;

(iii) a polynucleotide consisting of a base sequence which hybridizes to a base sequence of SEQ ID NO 6 under stringent conditions; and

(iv) a polynucleotide encoding a polypeptide consisting of an amino acid sequence which hybridizes to an amino acid sequence of SEQ ID NO 19 under stringent conditions.

In the 6^{th} polynucleotide, the base sequence set forth in SEQ ID NO: 6 or the amino acid sequence set forth in SEQ ID NO: 19, encodes for a partial MRPL17 gene (referred to as “truncated MRPL17”), encoding only 262 amino acids by deletion of the 263^{rd} amino acid.

The MRPL17 gene encodes a mitochondrial ribosomal protein.

The 6^{th} polynucleotide may be an isolated polynucleotide (referred to as a “6-1^{st} polynucleotide) having the base sequence set forth in SEQ ID NO: 31 and having a genetic map as shown in FIG. 6.

Referring to FIG. 6, the 6-1^{st} polynucleotide, derived from the 14^{th} chromosome of S. cerevisiae, encodes the truncated MRPL17 gene.

In one exemplary embodiment, the isolated polynucleotide may include a seventh polynucleotide (referred to as a “7^{th} polynucleotide”) selected from the following:

(i) a polynucleotide consisting of a base sequence having at least 90% identity to a base sequence of SEQ ID NO: 7; and

(ii) a polynucleotide consisting of a base sequence which hybridizes to SEQ ID NO: 7 under stringent conditions.

In the 7^{th} polynucleotide, the base sequence set forth in SEQ ID NO: 7 encodes for a partial YLR157C-B gene or YLRCTy1-1 gene. The 7^{th} polynucleotide is referred to as “partial fragment YLR157C-B.”

The YLR157C-B gene is a transposable element gene, which includes the retrotransposon TYA Gag and TYB Pol genes. The YLRCTy1-1 gene is a long terminal repeat (“LTR”) retrotransposon, which includes co-transcribed TYA Gag and TYB Pol genes, and encodes a protein involved in the structure and function of a virus-like particle (refer to Kim J. M., et al. (1998) Transposable elements and genome organization: a comprehensive survey of retrotransposons revealed by the complete Saccharomyces cerevisiae genome sequence. Genome Res. 8(5):464-78). These genes are involved in DNA-directed DNA polymerase activity, peptidase activity, protein binding, ribonuclease activity, RNA binding, and RNA-directed DNA polymerase activity.

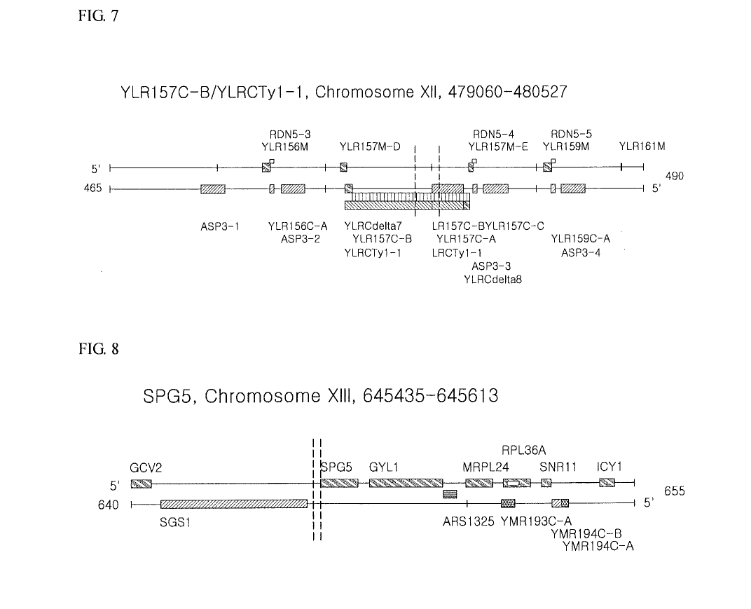

The 7^{th} polynucleotide has a genetic map as shown in FIG. 7. Referring to FIG. 7, the 7^{th} polynucleotide, derived from the 12^{th} chromosome of S. cerevisiae, encodes for the YLR157C-B and YLRCTy1-1 genes.

In one exemplary embodiment, the isolated polynucleotide may include an eighth polynucleotide (referred to as an “8^{th} polynucleotide”) selected from the following:

(i) a polynucleotide consisting of a base sequence having at least 90% identity to a base sequence of SEQ ID NO: 8; and

(ii) a polynucleotide consisting of a base sequence which hybridizes to a base sequence of SEQ ID NO: 8 under stringent conditions.

The 8^{th} polynucleotide has a genetic map as shown in FIG. 8. Referring to FIG. 8, the 8^{th} polynucleotide is derived from the 13^{th} chromosome of S. cerevisiae, and the base sequence of SEQ ID NO: 8 encodes a promoter site for the SPG5 gene. The 8^{th} polynucleotide is referred to as a “putative SPG5 promoter.”

The SPG5 gene encodes a protein necessary for growing microorganisms at a high temperature in stationary phase, and does not require a non-fermentable carbon source for growth.

The sequences and names of the isolated 1^{st} to 8^{th} polynucleotides and their corresponding examples, the 1-1^{st} to 6-1^{st} polynucleotides, which are described above, are shown in Table 2.

TABLE 2

| | | | | Base | A.A. |
|---|---|---|---|---|---|
| | Base | | | Sequence | Sequence |
| Name | Sequence | Gene | Name | (SEQ ID NO) | (SEQ ID NO) |
| 1-1^{st} polynucleotide | 26 | truncated M1H1 | 1^{st} polynucleotide | 1 | 14 |
| | | truncated MSN2 | — | 9 | 21 |
| 2-1^{st} polynucleotide | 27 | INO1 | 2^{nd} polynucleotide | 2 | 15 |
| | | truncated | — | 10 | 22 |
| | | VPS35 | | | |
| | | truncated SNA3 | — | 11 | 23 |
| 3-1^{st} polynucleotide | 28 | DOG1 | 3^{rd} polynucleotide | 3 | 16 |
| | | truncated | — | 12 | 24 |
| | | YHR045W | | | |
| 4-1^{st} polynucleotide | 29 | HAL1 | 4^{th} polynucleotide | 4 | 17 |
| | | truncated | — | 13 | 25 |
| | | AIM45 | | | |
| 5-1^{st} polynucleotide | 30 | TRP1 | 5^{th} polynucleotide | 5 | 18 |
| 6-1^{st} polynucleotide | 31 | truncated | 6^{th} polynucleotide | 6 | 19 |
| | | MRPL17 | | | |
| 7^{th} polynucleotide | 7 | Partial fragment | — | 7 | — |
| | | YLR157C-B | | | |
| 8^{th} polynucleotide | 8 | putative SPG5 | — | 8 | — |
| | | promoter | | | |

The isolated polynucleotides encode proteins that increase the alcohol tolerance of host cells.

The alcohol tolerance of a host cell may be determined by a specific growth rate in the minimum inhibitory concentration (MIC) of the host cell. The growth rate may be measured by a colony forming unit (CFU), a final cell density, or a decreased rate of lag time.

In one exemplary embodiment, the alcohol tolerance may be expressed as the “specific growth rate” in the MIC, where the “specific growth rate” may be expressed by the following Equation (1), representing a cell growth rate per unit time.

$$specific growth rate ( {h}^{- 1} ) = \frac{1}{x} \frac{d [ x ]}{d t} \qquad ( 1 )$$

In Equation (1), x is the cell concentration as measured in grams per liter (g/L), and t is time.

As used herein, the MIC means a minimum concentration of a material that inhibits the growth and survival of at least 99% of existing microbial colonies, that is, the minimum concentration causing the induction of apoptosis. For example, in the case of wild-type S. cerevisiae, the MIC may be about 5% for ethanol and about 1% for isobutanol.

Accordingly, when the polynucleotide is overexpressed in a host cell utilized for alcohol fermentation, usually in fermentation yeast, the alcohol tolerance of the host cell is increased.

Examples of the alcohol produced by the host cell include ethylene glycol, propylene glycol, polyethylene glycol, polypropylene glycol, polyethylene glycopropylene glycol), 1,3-propanediol, 1,2-butanediol, 2,3-butanediol, 1,4-butanediol, 1,6-hexanediol, pinacol, glycerol, neopentylglycol, pentaerythritol, mezo-hydrobenzoin, 1,2-cyclopentanediol, 1,2-cyclohexanediol, methanol, ethanol, isopropanol, n-propanol, n-butanol, isobutanol, sec-butanol, tert-butanol, n-pentanol, isopentanol, tert-pentanol, cyclopentanol, cyclohexanol, n-hexanol, n-heptanol, n-octanol, n-nonanol, n-decanol, phenoxyethanol, benzylalcohol, diphenyl carbinol, tetraphenylcarbinol, and mixtures thereof.

In one exemplary embodiment, the alcohol may be ethanol or isobutanol.

The various polynucleotides described herein may be recombinant polynucleotides. The recombinant polynucleotides may be synthetic polynucleotides or other polynucleotides engineered in vitro. The recombinant polynucleotide may be used to produce gene products in cells or other biological systems. For example, a cloned polynucleotide may be inserted into a suitable expression vector (e.g., a plasmid), and then the expression vector may be used to transform the suitable host cell. The host cell containing the recombinant polynucleotide is referred to as a “recombinant host cell.” When a gene is expressed in the recombinant host cell, a “recombinant protein” is produced. The recombinant polynucleotide may also have non-coding regions (or sequences), e.g., a promoter, a replication origin, a ribosome-binding site, and the like.

2. Vector

According to another exemplary embodiment, a vector containing the isolated polynucleotide is provided.

The term “vector” refers to a nucleic acid construct having a polynucleotide sequence operably linked to an expression regulatory sequence. The term “operably linked” refers to the association between of nucleic acid sequences on a single nucleic acid fragment so that the function of one part (e.g., capability of regulating transcription) is regulated by the other part (e.g., transcription of a sequence). For example, a promoter is operably linked with a coding sequence when it is capable of regulating the expression of that coding sequence (i.e., that the coding sequence is under the transcriptional control of the promoter) or a ribosome binding site is operably linked to a coding sequence if it is positioned so as to facilitate translation. Coding sequences can be operably linked to regulatory sequences in a sense or antisense orientation.

Accordingly, when a polynucleotide expression regulatory sequence (e.g., a promoter or other transcription regulatory sequences) is linked to a desired polynucleotide sequence (e.g., natural or recombinant polynucleotide) by a functional connection, the polynucleotide is operably linked to the expression regulatory sequence, thereby allowing the expression regulatory sequence to direct the transcription of the polynucleotide.

The expression regulatory sequence or promoter is an expression regulatory sequence directing the transcription of the polynucleotide, which may be an extrinsic or an intrinsic polynucleotide. The promoter has a nucleic acid sequence, such as a polymerase-binding site, adjacent to the transcription start site. In addition, the promoter may also include a terminal enhancer or repressor element.

Available vectors include, but are not limited to, bacteria, plasmids, phages, cosmids, episomes, viruses and insertable DNA fragments. The term “plasmid” refers to a circular, extra-chromosomal, double-stranded DNA molecule typically capable of autonomous replication within a suitable host cell and into which foreign DNA has been inserted. The plasmid is capable of inserting the foreign DNA into a host genome.

The vector may produce a protein or peptide encoded by a polynucleotide described herein by introduction into a host cell.

Examples of promoters suitable for use in yeast include, but are not limited to, GAPDH, PGK, ADH, PHO5, GAL1 and GAL10. The vector may also include an additional regulatory sequence. Examples of suitable regulatory sequences include a Shine-Dalgarno sequence found in the replicase gene of phage MS-2, and a Shine-Dalgarno sequence found in cII of bacteriophage λ. Moreover, the expression vector may include a suitable marker that may be used to select transfected host cells.

Examples of vectors capable of expression and genetic recombination in fermentation microorganisms such as yeast include, but are not limited to, 2 micron, pBM272, pBR322-6, pBR322-8, pCS19, pDW227, pDW229, pDW232, pEMBLYe23, pEMBLYe24, pEMBLYi21, pEMBLYi22, pEMBLYi32, pEMBLYr25, pFL2, pFL26, pFL34, pFL35, pFL36, pFL38, pFL39, pFL40, pFL44L, pFL44S, pFL45L, pFL45S, pFL46L, pFL46S, pFL59, pFL59+, pFL64−, pFL64+, pG6, pG63, pGAD10, pGAD424, pGBT9, pGKl2, pJRD171, pKD1, pNKY2003, pNKY3, pNN414, pON163, pON3, pPM668, pRAJ275, pRS200, pRS303, pRS304, pRS305, pRS306, pRS313, pRS314, pRS315, pRS316, pRS403, pRS404, pRS405, pRS406, pRS413, pRS414, pRS415, pRS416, pRS423, pRS424, pRS425, pRS426, pRSS56, pSG424, pSKS104, pSKS105, pSKS106, pSZ62, pSZ62, pUC-URA3, pUT332, pYAC2, pYAC3, pYAC4, pYAC5, pYAC55, pYACneo, pYAC-RC, pYES2, pYESHisA, pYESHisB, pYESH is C, pYEUra3, rpSE937, YCp50, YCpGAL0, YCpGAL1, YCplac111, YCplac22, YCplac33, YDp-H, YDp-K, YDp-L, YDp-U, YDp-W, YEp13, YEp213, YEp24, YEp351, YEp352, YEp353, YEp354, YEp355, YEp356, YEp356R, YEp357, YEp357R, YEp358, YEp358R, YEplac112, YEplac181, YEplac195, YIp30, YIp31, YIp351, YIp352, YIp353, YIp354, YIp355, YIp356, YIp356R, YIp357, YIp357R, YIp358, YIp358R, YIp5, YIplac128, YIplac204, YIplac211, YRp12, YRp17, YRp7, pAL19, paR3, pBG1, pDBlet, pDB248X, pEA500, pFL20, pIRT2, pIRT2U, pIRT2-CAN1, pJK148, pJK210, pON163, pNPT/ADE1-3, pSP1, pSP2, pSP3, pSP4, pUR18, pUR19, pZA57, pWH5, pART1, pCHY21, pEVP11, REP1, REP3, REP4, REP41, REP42, REP81, REP82, RIP, REP3X, REP4X, REP41X, REP81X, REP42X, REP82X, RIP3X/s, RIP4X/s, pYZ1N, pYZ41N, pYZ81N, pSLF101, pSLF102, pSLF104, pSM1/2, p2UG, pART1/N795, and pYGT. In one example, the vector may be plasmid pRS424.

3. Host Cell

In still another exemplary embodiment, a host cell capable of producing alcohol when incubated in a monosaccharide-containing nutrient source, and having one or more kinds of overexpressed polypeptides for increasing the alcohol tolerance of the host cell is provided. In another exemplary embodiment, the host cell demonstrates increased monosaccharide uptake rate when incubated in a monosaccharide-containing nutrient source, and is capable of being grown in a minimal medium.

Due to high alcohol tolerance, the host cell can survive at a high rate even in high concentrations of alcohol when incubated in a monosaccharide-containing nutrient source such as glucose or galactose. Thus, the host cell can have tolerance to various inhibitors that inhibit the production of fermentation products in high-capacity industrial fermentation processes, and which prevent the cell growth inhibition phenomenon in high concentrations of ethanol. As a result, the process is very economical since the alcohol production yield is increased and the availability of the host cell is increased.

In one exemplary embodiment, the polypeptide is encoded by an isolated polynucleotide including one selected from the group consisting of: (a) a polynucleotide consisting of a base sequence having at least 90% identity to a base sequence selected from SEQ ID NOs: 1 to 8; (b) a polynucleotide encoding a polypeptide consisting of an amino acid sequence having at least 90% identity to an amino acid sequence selected from SEQ ID NOs: 14 to 19; (c) a polynucleotide consisting of a base sequence which hybridizes to a base sequence selected from SEQ ID NOs: 1 to 8 under stringent conditions; and (d) a polynucleotide encoding a polypeptide consisting of an amino acid sequence which hybridizes to an amino acid sequence selected from SEQ ID NOs: 14 to 19 under stringent conditions.

In one exemplary embodiment, the polypeptide may include an amino acid sequence having at least 90% identity to an amino acid sequence of SEQ ID NO: 14, or an amino acid sequence which hybridizes to an amino acid sequence of SEQ ID NO: 14 under stringent conditions.

In another exemplary embodiment, the polypeptide may include an amino acid sequence having at least 90% identity to an amino acid sequence of SEQ ID NO: 15, or an amino acid sequence which hybridizes to an amino acid sequence of SEQ ID NO: 15 under stringent conditions.

In yet another exemplary embodiment, the polypeptide may be encoded by a polynucleotide consisting of a base sequence having at least 90% identity to a base sequence of SEQ ID NO: 26 or 27.

The host cells are capable of producing alcohol when incubated in a monosaccharide-containing nutrient source, and may be selected from, but are not limited to, bacteria, fungi or yeasts. The host cell may also provide a suitable cell environment for expressing the polynucleotide described herein.

Examples of the host cells include, but are not limited to, those selected from the group consisting of Saccharomyces cerevisiae, Klebsiella oxytoca P2, Brettanomyces curstersii, Saccharomyces uvzrun, Candida brassicae, Sarcina ventriculi, Zymomonas mobilis, Kluyveromyces marxianus IMB3, Clostridium acetobutylicum, Clostridium beijerinckii, Kluyveromyces fragilis, Brettanomyces custersii, Clostriduim aurantibutylicum and Clostridium tetanomorphum.

In some embodiments, the host cell is a yeast. The yeast may be selected from the group consisting of the genera Saccharomyces, Pachysolen, Clavispora, Kluyveromyces, Debaryomyces, Schwanniomyces, Candida, Pichia, and Dekkera.

In one exemplary embodiment, the host cell described herein may exhibit at least about 1%, about 2%, about 5%, about 8%, about 10%, about 12%, about 15% or about 20% increase in the specific growth rate (h⁻¹) in the MIC, as compared to the wild-type S. cerevisiae. In this case, the MIC may be about 5% for ethanol or about 1% for isobutanol.

In another exemplary embodiment, the host cell may exhibit at least about 5%, about 10%, about 15%, about 20%, about 25%, about 30%, about 35%, about 40%, about 45% or about 50% increase in volumetric productivity of ethanol (g/L/h), as compared to the wild-type S. cerevisiae under the same conditions for incubation. In this case, the ethanol volumetric productivity refers to the time required to produce the maximum concentration of ethanol by consuming the given substrate(s).

In yet another exemplary embodiment, the host cell may exhibit at least about 5%, about 10%, about 15%, about 20%, about 25%, about 30%, about 35%, about 40%, about 45% or about 50% increase in specific ethanol production rate (g ethanol/g dry cell/h), as compared to the wild-type S. cerevisiae under the same conditions for incubation. In this case, the “specific ethanol production” rate refers to the rate of time for consuming given substrate(s) to convert into ethanol, for a unit time per unit cell.

In an exemplary embodiment, the host cell is an overexpressing strain exhibiting an enhancement in expression of a predetermined polypeptide. Here, “enhancement” means an increase in intracellular activity or concentration of a protein encoded by the polynucleotide.

By overexpression, the activity or concentration of a polypeptide is increased by about 10%, about 25%, about 50%, about 75%, about 100%, about 150%, about 200%, about 300%, about 400% or about 500% and as much as about 1000% or about 2000%, as compared to that of a polypeptide present in the wild-type strain, which may be the strain of S. cerevisiae.

In one exemplary embodiment, the overexpression of the polypeptide may be achieved by increasing the number of copies of a gene encoding a corresponding protein. A gene or a gene construct may be present in a plasmid replicable in multi-copies, or integrated into a chromosome.

In another exemplary embodiment, overexpression may be achieved by regulating the gene encoding the corresponding protein through the control of a gene regulatory sequence that is not naturally present in the gene, such that it is recombinantly inserted into the gene. For example, overexpression may be brought about through transformation of a promoter, a regulatory region or a ribosome-binding site into a constructive gene. Overexpression may also be achieved using a gene encoding a transformed protein having a specific activity that is higher than a wild-type protein of the host cell, or an allele thereof, or by changing the composition of the growth media and/or the incubation process.

In addition, overexpression may also be achieved by methods known in the art (refer to Eikmanns et al. (Gene 102, 93-98 (1991)); EP 0 472 869; LaBarre et al. (Journal of Bacteriology 175, 1001-1007 (1993)); and WO 96/15246, the teachings of which are incorporated herein in their entirety).

Various methods of introducing the polynucleotide or vector into the host cell are also known in the art (refer to Molecular Cloning: A Laboratory Manual, 2nd Edition, Sambrook et al. Cold Spring Harbor Laboratory Press, (1989)).

Examples of methods used for introducing the polynucleotide or vector into the host cell include calcium phosphate transfection, DEAE-dextran-mediated transfection, transvection, microinjection, cationic lipid-mediated transfection, electrophoration, transduction, scrape loading, ballistic transduction, or transfection. Further, for these methods, various expression systems including chromosomes, episomes and virus-derived systems, bacterial plasmids, bacteriophages, transposons, enzyme episomes, insertion elements, enzyme chromosome elements and virus-derived vectors may be used.

The expression systems regulate expression and may also include a regulatory region. In general, to produce a polypeptide in a host cell, all systems or vectors that can maintain, multiply, or express the polynucleotide, may be used. In exemplary embodiments, the overexpression method is a method of using a plasmid vector.

In another exemplary embodiment, the host cells according to the inventive concept are strains exhibiting excellent alcohol tolerance, and were deposited in the Genebank of the Korea Research Institute of Bioscience and biotechnology (KRIBB; Yuseong-gu, Daejeon, Korea) on 16 Mar. 2009. Accession numbers, names, and names of the isolated polynucleotides included in the respective host cells are shown in Table 3.

TABLE 3

| Accession | | Isolated |
|---|---|---|
| No. | Name | Polynucleotide |
| KCTC11476BP | S. cerevisiae CEN.PK2-1D/pRS424-MSN2/MIH1 | 1-1^{st} polynucleotide |
| KCTC11477BP | S. cerevisiae CEN.PK2-1D/pRS424-INO1 | 2-1^{st} polynucleotide |
| KCTC11478BP | S. cerevisiae CEN.PK2-1D/pRS424-DOG1 | 3-1^{st} polynucleotide |
| KCTC11479BP | S. cerevisiae CEN.PK2-1D/pRS424-HAL1 | 4-1^{st} polynucleotide |
| KCTC11480BP | S. cerevisiae CEN.PK2-1D/pRS424-TRP1 | 5-1^{st} polynucleotide |
| KCTC11481BP | S. cerevisiae CEN.PK2-1D/pRS424-MRPL17 | 6-1^{st} polynucleotide |
| KCTC11482BP | S. cerevisiae CEN.PK2-1D/pRS424-YLR157C-B | 7^{th} polynucleotide |
| KCTC11483BP | S. cerevisiae CEN.PK2-1D/pRS424-SPG5p | 8^{th} polynucleotide |

4. Method of Producing Bioalcohol

According to another exemplary embodiment, a method of producing bioalcohol by incubating the host cell in a monosaccharide-containing nutrient source and producing alcohol through fermentation is provided.

Fermentation, as expressed by the following reaction formulae, is the conversion of monosaccharides produced by saccharification into alcohol through fermentation by microorganisms.

C₆H₁₂O₆→2C₂H₅OH+2CO₂ (2)

3C₅H₁₀O₅→5C₂H₅OH+5CO₂ (3)

In one exemplary embodiment, the method of producing bioalcohol comprises: engineering a host cell to overexpress one or more isolated polynucleotides encoding a polypeptide for increasing alcohol tolerance of the host cell, wherein the isolated polypeptide is at least one selected from the group consisting of: (a) a polynucleotide consisting of a base sequence having at least 90% identity to a base sequence selected from SEQ ID NOs: 1 to 8, (b) a polynucleotide encoding a polypeptide consisting of an amino acid sequence having at least 90% identity to an amino acid sequence selected from SEQ ID NOs: 14 to 19, (c) a polynucleotide consisting of a base sequence which hybridizes to a base sequence selected from SEQ ID NOs: 1 to 8 under stringent conditions, and (d) a polynucleotide encoding a polypeptide consisting of an amino acid sequence which hybridizes to an amino acid sequence selected from SEQ ID NOs: 14 to 19 under stringent conditions; incubating the host cell in a monosaccharide-containing nutrient source under conditions suitable for producing alcohol; and producing alcohol through fermentation.

The engineering of the host cell may be performed by any method of overexpressing one or more polypeptides listed above, for example, a method of using an expression vector such as a plasmid. For example, the engineering may be performed by inserting an isolated polynucleotide encoding the polypeptide into a vector, amplifying the vector, and inserting the vector into the host cell.

In one exemplary embodiment, the host cell may be a yeast cell which is derived from S. cerevisiae.

In one exemplary embodiment, the monosaccharide may include at least one selected from the group consisting of glucose, galactose, galactose derivatives, 3,6-anhydrogalactose, fucose, rhamnose, xylose, glucuronic acid, arabinose and mannose, or a mixture of glucose and galactose.

The monosaccharide may be a hydrolyte of sugar biomass, woody biomass or algae biomass.

In another exemplary embodiment, the bioalcohol is a fuel produced from a biomass. The bioalcohol may be, but is not limited to, ethanol, propanol, isopropanol, butanol, isobutanol, acetone, ethylene, propylene, fatty acid methyl ester, or a mixture thereof.

In one exemplary embodiment, before fermentation, saccharification may be required to produce a monosaccharide-containing nutrient source. The saccharification is a hydrolysis operation of a biomass or polysaccharide into monosaccharides using a hydrolysis catalyst such as sulfuric acid, or an enzyme such as hydrolase.

The saccharification and fermentation may be performed in separate reaction vessels through separate hydrolysis and fermentation (SHF) processes, or in one reaction vessel through a simultaneous saccharification and fermentation (SSF) process.

The SHF process may be performed under optimized conditions for the respective saccharification and fermentation processes, but may create inhibition of enzymatic hydrolysis between an intermediate product and a final product. Thus, more enzymes are needed to overcome this problem, which is uneconomical. For example, as an intermediate product, cellobiose is converted into a final product, glucose, during the saccharification of cellulose, the glucoses are accumulated, thereby inducing inhibition of the hydrolysis between the intermediate product and the final product, resulting in termination of the reaction.

In comparison, in the SSF process, as soon as glucose is produced during saccharification, yeast consumes the glucose through fermentation and thus glucose accumulation in a reaction vessel can be minimized. As a result, inhibition driven by a final product, which can occur in the SHF process, can be prevented, and hydrolysis mediated by a hydrolase (enzyme) can be enhanced. Further, the SSF process can reduce production costs due to lower equipment costs and lower inputs of enzyme, and also lessen the risk of contamination due to ethanol present in the reaction vessel.

Conditions for the fermentation are not particularly limited. In one exemplary embodiment, fermentation may be performed by stirring under conditions comprising: an initial glucose concentration of about 2 to about 30% (w/v), a temperature of about 25 to about 37° C., pH of about 5.0 to about 8.0, and a stirring rate of about 100 to about 250 rpm.

Additional operations and/or other processes may be selected by those skilled in the art as the occasion demands. For example, the operations or processes may include pretreatment of the biomass through grinding or hydrolysis to be suitable for saccharification, or purification of a fermented solution yielded by the fermentation according to the method known in the art.

5. Method of Selecting Gene Exhibiting Alcohol Tolerance

According to another exemplary embodiment, a method of selecting a gene exhibiting an increase in alcohol tolerance when overexpressed in a yeast cell is provided, which includes the following operations.

Operation A: A yeast genomic library (e.g., S. cerevisiae) is constructed using a multi-copy plasmid.

In operation A, the method of constructing the genomic library comprises (i) digesting genomic DNA of the wild-type strain of S. cerevisiae using restriction enzymes, (ii) introducing a digested DNA fragment into a multi-copy plasmid, and (iii) amplifying the plasmid. In operation A, the yeast may be S. cerevisiae strain CEN. PK2-ID, and the multi-copy plasmid may be pRS424.

Operation B: The genomic library constructed in operation A is transformed into a yeast cell, thereby constructing a library of the transformed yeast (referred to as the “test strain”) in which all genes are overexpressed. The transformation operation may be performed by a conventional method (refer to Ito, H., Y. Fukuoka, K. Murata, A. Kimura (1983) Transformation of intact yeast cells treated with alkali cations, J. Bacteriol. 153, 163-168; incorporated herein in its entirety).

Operation C: After confirming the MIC of the test strain, the cells of the test strain are plated and incubated on agar plates containing various isobutanol gradients. Subsequently, cells grown in a relatively high concentration alcohol are selected, resulting in a library stock. The alcohol MIC of the test strain may be about 5% for ethanol or about 1% for isobutanol.

Operation D: A liquid minimal medium (SC media; Synthetic Complete media) containing isobutanol at the MIC is inoculated with the library stock, and followed by serial subculture in fresh medium having the same isobutanol concentration in order to enrich the culture with a lower concentration of inoculation. The serial subculture may be repeated about 5 to about 10 times. The minimal medium may contain about 100 to about 300 g/L of glucose, and/or about 20 to about 80 g/L of galactose. After the enrichment, a predetermined amount of the culture is diluted and plated on an agar plate containing isobutanol in the MIC for incubation. Then, big colonies are selected from the plate.

Operation E: Alcohol tolerance tests are performed on the selected big colonies, so that a strain exhibiting excellent alcohol tolerance is selected. Plasmids are isolated from the selected transformed yeast cells, and then the inserted yeast genomic sequence introduced into the isolated plasmid is identified using the known gene sequence at both sides of the restriction enzymes used for cloning. In this case, the gene sequence may be confirmed using Gel documentation (gel doc) or Hydra.

In one example, the selection method may further include the following operations after operation E.

Operation F: Both terminal sequences of the insert introduced into the plasmid are compared to the yeast gene sequence to confirm the exact gene introduced into the multi-copy plasmid.

Operation G: The plasmid containing the confirmed gene is transformed into a yeast cell again to confirm if genes contained in the isolated plasmid cause the alcohol tolerance effect.

Herein, by the above-described method, a total of 8 genes involved in alcohol tolerance were discovered.

The inventive concept will be described in more detail below with reference to various examples.

### Construction Example 1

### 1-1. Construction of Genomic Library

To construct the genomic library of strain CEN.PK2-1D of S. cerevisiae (MATalpha; ura3-52; trp1-289; leu2-3_{—}112; his3 D1; MAL2-8C; SUC2), genomic S. cerevisiae DNA was first fragmented by sonication. Then, genomic fragments having sizes of 2 to 4 kilobases (kb) were selected on an agarose gel.

Subsequently, the multi-copy plasmid (pRS424) was digested with the restriction enzyme BamHI, and followed by a fill-in reaction to create blunt ends.

The selected genomic fragment was inserted into the blunt-ended pRS424 by ligation using the enzyme T4 DNA ligase. The plasmid containing the genomic fragment (e.g. isolated polynucleotide), constructed as described above, was transformed into E. coli and then amplified to construct the genomic DNA library.

### 1-2. Construction of Transformed Yeast Library

S. cerevisiae strain CEN.PK2-1D was incubated in yeast/peptone/dextrose (“YPD”) liquid media (containing 10 g of Yeast extract/L, 20 g of Peptone/L, 20 g of Dextrose/L). Subsequently, the plasmid library constructed in Construction Example 1-1 was transformed into the CEN.PK2-1D strain using an Alkali-Cation Yeast Kit (MP Biomedicals), resulting in a transformed yeast library.

### 1-3. Preparation of Library Stock

The transformed yeast cells (referred to as the “test strain”) were plated on solid minimal media plates and incubated for about 48 hours at about 30° C. After that, colonies grown on the plates were harvested, resulting in a library stock. The solid minimal media plates, containing 6.7 g/L of YNB, 20 g/L of glucose, 6.2 mg/L of CSM-Leu, 0.01% (w/v) Leucine, and 0.2% (w/v) Uracil without Tryptophan, were prepared by sterilizing the media with 2% (w/v) agar at high temperature, and pouring them into large agar plates (SPL Co.).

### 1-4. Sequence Analysis and Preparation of Host Cell

Strains exhibiting excellent alcohol tolerance were selected from amongst the isolated colonies through an ethanol tolerance test according to the following protocol. Plasmids were isolated from the selected 8 strains using a Zymoprep kit (Zymo research). To confirm the sequence of the isolated polynucleotide contained in the plasmid, the sequence of the cloned gene was analyzed using a sequencing primer prepared based on a sequence of the cloned plasmid gene.

[Protocol for Ethanol Tolerance Test]

Strains were incubated in a 4-baffle flask (250 ml) containing minimal medium at the final volume of 50 ml for, 2 days at 30° C. One ml of each culture for the 150 g/L ethanol viability test, and 2 ml of each culture for the 170 g/L ethanol viability test were centrifuged, and then the pellets were washed with distilled water. The pellets were centrifuged again, and then suspended in respective 15 ml conical tubes (SPL) containing 5 ml of 20 g/L glucose minimal medium (SC-Trp) for incubation. The cultures were incubated for up to 2-days at a stirring rate of 200 rpm, and at a temperature of 30° C. From each culture, samples of at least 100 μl up to 500 μl were removed every 3 hours, and streaked on solid minimal media diluted in moderation.

### Examples 1-8

Eight strains were selected by the selection according to Construction Example 1, and 8 plasmids were successfully isolated from each strain. The genomic sequence of the isolated polynucleotide contained in each plasmid was confirmed. To confirm the effects of overexpression for each of these sequences, the isolated plasmids were re-introduced into the S. cerevisiae CEN.PK2-1D parent strain using an EZ-Yeast Transformation Kit (MP Biomedicals), resulting in the host cells of Examples 1 to 8. The SEQ ID NOs and the names of the isolated polynucleotides included in the respective plasmids introduced into the host cells of Examples 1 to 8 are shown in Table 4.

TABLE 4

| Example | SEQ ID NO: | Name |
|---|---|---|
| 1 | 26 | 1-1^{st} polynucleotide |
| 2 | 27 | 2-1^{st} polynucleotide |
| 3 | 28 | 3-1^{st} polynucleotide |
| 4 | 29 | 4-1^{st} polynucleotide |
| 5 | 30 | 5-1^{st} polynucleotide |
| 6 | 31 | 6-1^{st} polynucleotide |
| 7 | 7 | 7^{th} polynucleotide |
| 8 | 8 | 8^{th} polynucleotide |

### Experimental Example 1

### Cell Growth Rate in 5% (w/v) Ethanol Liquid Medium

Five % (w/v) ethanol-containing minimal medium was inoculated with strains of Examples 1 to 8 for stationary culture at 30° C. C. For the stationary culture, a 15 ml falcon tube and a minimal medium (SC medium) were used. Initial inoculation was carried out using an overnight culture at a low cell density (OD₆₀₀: about 0.05) in the total volume of about 5 ml.

Every 12 hours, samples were taken from each culture, and cells were isolated and washed for measurement of optical density to analyze cell growth rate. The optical density (OD) was measured using a UV spectrophotometer (A600 nm).

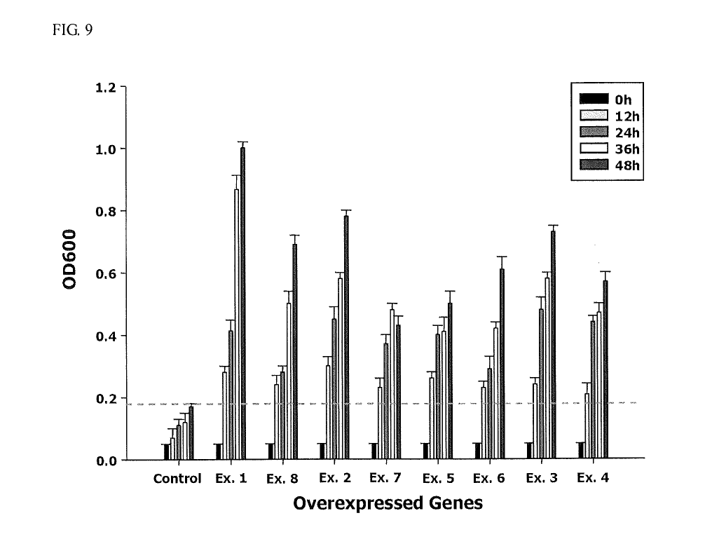

The results are shown in FIG. 9. It can be seen from FIG. 9 that all host cells of Examples 1 to 8 show higher cell growth rates than the control group, wild-type S. cerevisiae yeast.

### Experimental Example 2

### Cell Growth Rate and Ethanol Volumetric Productivity in 1% (w/v) Isobutanol-Containing Liquid Medium

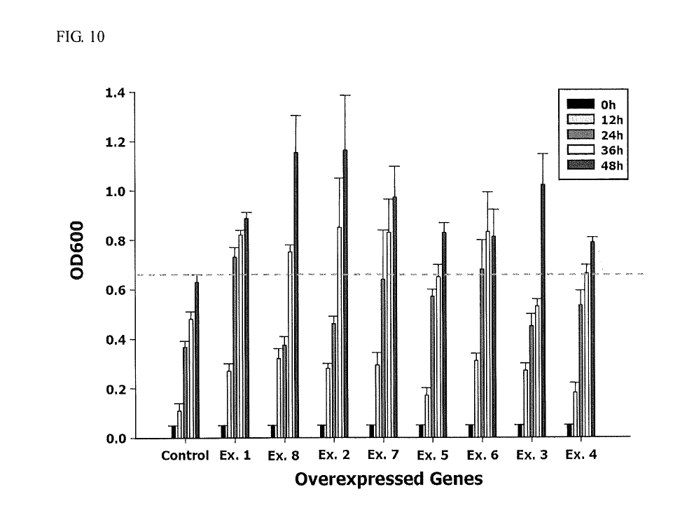

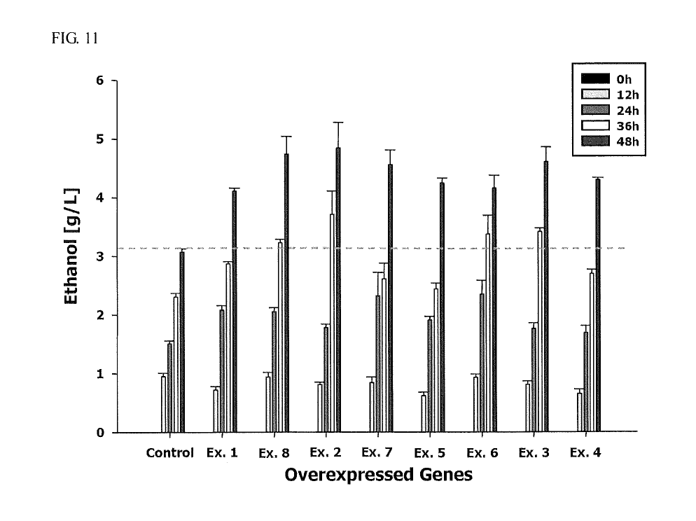

The cell growth rate was analyzed by the same method described in Experimental Example 1, except a culture medium containing 1% (w/v) isobutanol, instead of 5% (w/v) ethanol, was used. The results are shown in FIG. 10.

It can be seen from FIG. 10 that all host cells of Examples 1 to 8 show higher cell growth rates than the control group, S. cerevisiae wild-type yeast.

In addition, the ethanol volumetric productivity was analyzed by gas chromatography (GC), and the results are shown in FIG. 11. It can be seen that all host cells of Examples 1 to 8 show a significant increase in ethanol volumetric productivity, as compared to the control group, the wild-type yeast S. cerevisiae.

### Experimental Example 3

### Viability According to Ethanol Concentration in Solid Medium

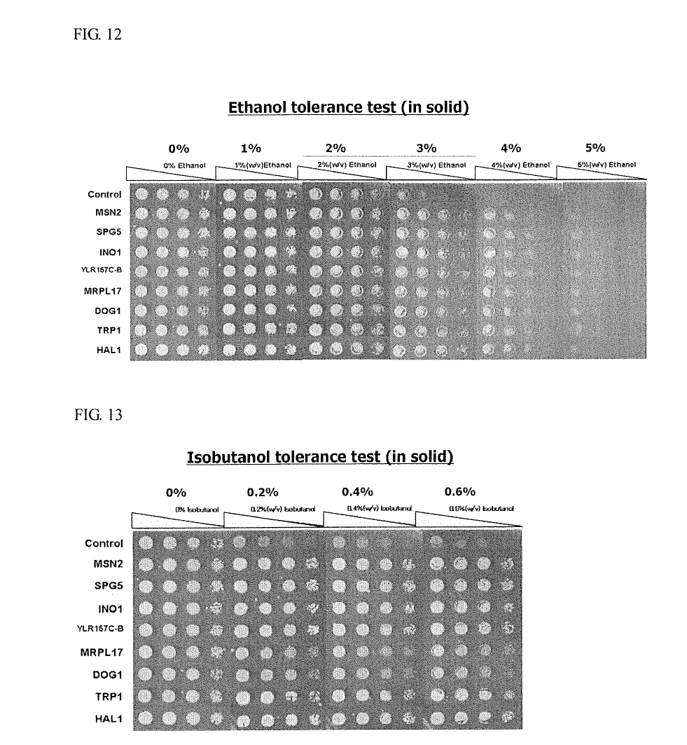

The strains of Examples 1 to 8 were incubated in minimal solid media (SC media) containing ethanol at a concentration of 0, 1, 2, 3, 4 and 5% (w/v), and 2% (w/v) glucose, respectively. Cells were serially diluted three times by a fifth and then patched on a plate from the left (standard: OD600=1) to the right sides. The results are shown in FIG. 12.

It can be seen from FIG. 12 that the host cells of Examples 1 to 8 show an increase in viability as compared to the control group, the wild-type yeast S. cerevisiae. Particularly, the yeast strains into which the isolated polynucleotides of Examples 1, 2, 4, 7 and 8 were introduced show a strong tolerance to ethanol.

### Experimental Example 4

### Viability According to Isobutanol Concentration in Solid Medium

Cell viabilities were analyzed by the same method described in Experimental Example 3, except plates containing isobutanol at a concentration of 0, 0.2, 0.4 and 0.6% were used instead of ethanol, and the results are shown in FIG. 13.

It can be seen that the host cells of Examples 1 to 8 show increases in viability as compared to the control group, the wild-type yeast. Particularly, the yeast strains into which the polynucleotides of Examples 1, 2, 4, 7 and 8 are introduced show a strong tolerance to isobutanol.

### Experimental Example 5

### Fermentation Test in 5% Ethanol and 10% Glucose

The yeast strains of Examples 1 to 4 were fermented in mixed liquid media containing 5% (w/v) ethanol and 10% (w/v) glucose. Each yeast strain was incubated in a 250 ml glass flask containing 50 ml of minimal medium (SC media), and grown to a high cell density.

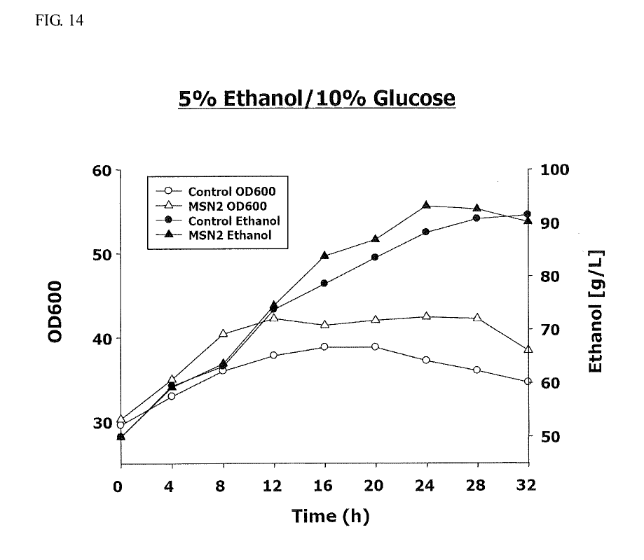

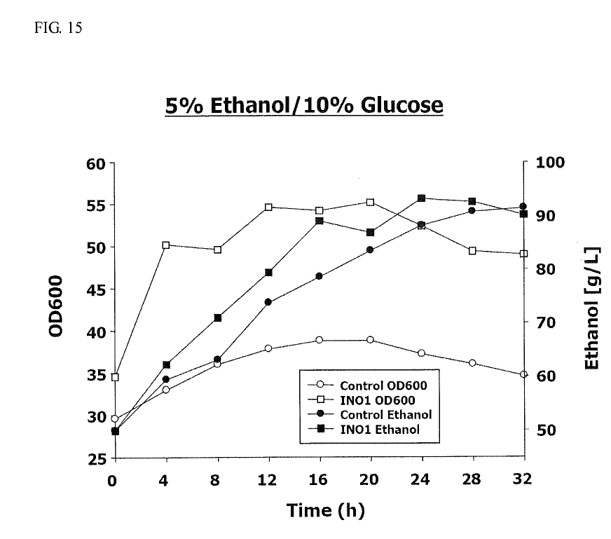

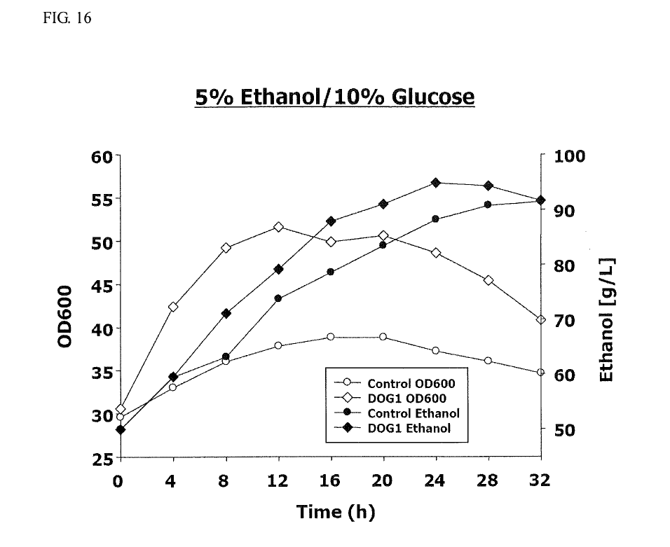

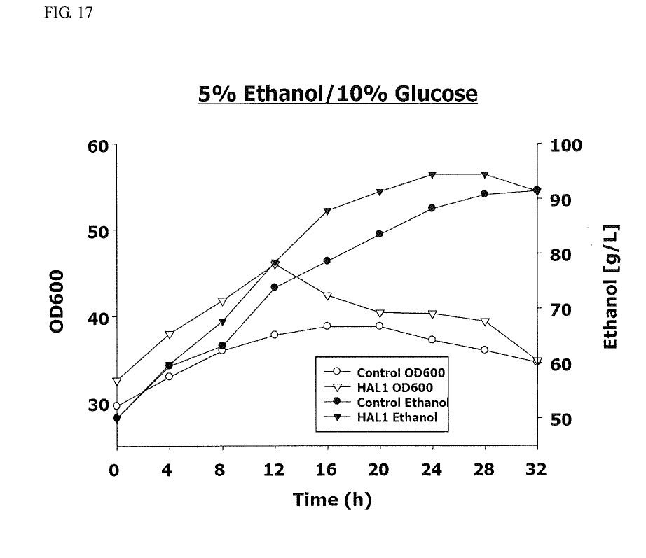

Viability was analyzed by a spectrophotometer (OD600), and the ethanol volumetric productivity was analyzed by gas chromatography (GC). To analyze the ethanol volumetric productivity, a standard solution was prepared by mixing 100 g/L of 1-propanol and 100 g/L of ethanol in the ratio of 1:1 (v/v), and then the value obtained using 100 g/L of ethanol and 100 g/L of 1-propanol was set as an internal standard. Then, samples were mixed with 100 g/L of 1-propanol in the ratio of 1:1 (v/v), and injected into the gas chromatograph. The ethanol volumetric productivity per unit sample was calculated in g/L. The results are shown in FIGS. 14 to 17.

In addition, to confirm whether the specific growth rate (h⁻¹) and ethanol volumetric productivity (g/L/h) were increased or not, data obtained by the fermentation test were calculated using parameters, and the results are shown in Table 5.

TABLE 5
[5% ethanol, 10% glucose test]

| | Control | Example 1 | Example 2 | Example 3 | Example 4 |
|---|---|---|---|---|---|
| Specific growth rate (h⁻¹) 0-12 h | 0.020 | 0.028 | 0.038 | 0.044 | 0.029 |
| Volumetric productivity (g/L/h) | 1.783 | 2.108 | 2.435 | 2.364 | 2.358 |
| 0-16 h | | | | | |

Referring to FIGS. 14 to 17 and Table 5, it can be seen that the strains of Examples 1 to 4 show increases in viability as compared to the control group, the wild-type strain, indicating an increase in alcohol tolerance. It can be also seen that the time for producing 90 g/L ethanol is shorter in the strains of Examples 1 to 4 (20 to 24 h) than in the control strain (28 to 32 h), indicating an increase in alcohol volumetric productivity.

Particularly, it can be seen that the strain of Example 2 shows about a 90% increase in specific growth rate and about a 37% increase in ethanol volumetric productivity as compared to the control strain. It can be also seen that the strain of Example 3 shows about a 100% or more increase in the specific growth rate, which is 0.044 (h⁻¹), as compared to the control strain (0.02).

### Experimental Example 6

### Fermentation Test in 5% ethanol and 20% glucose

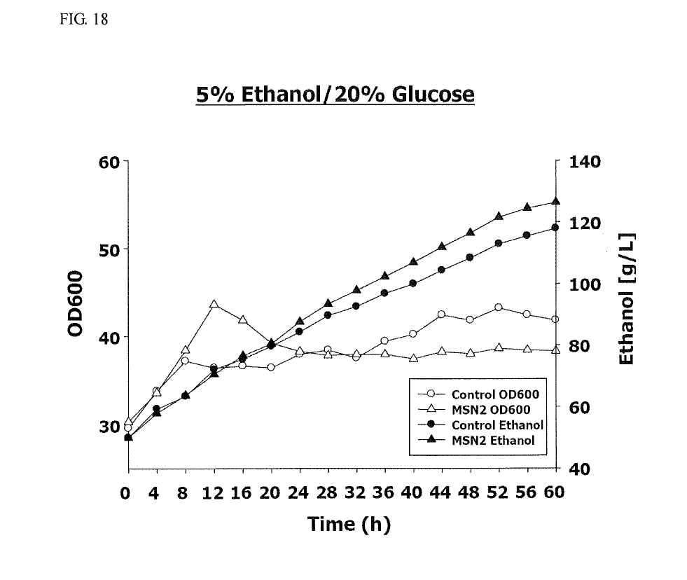

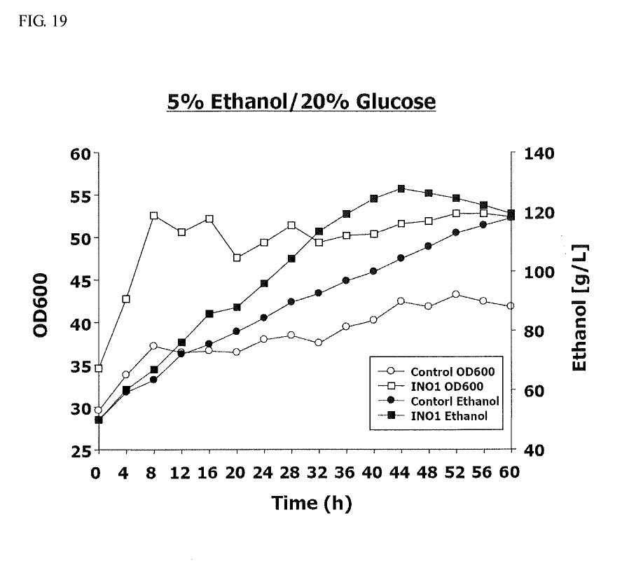

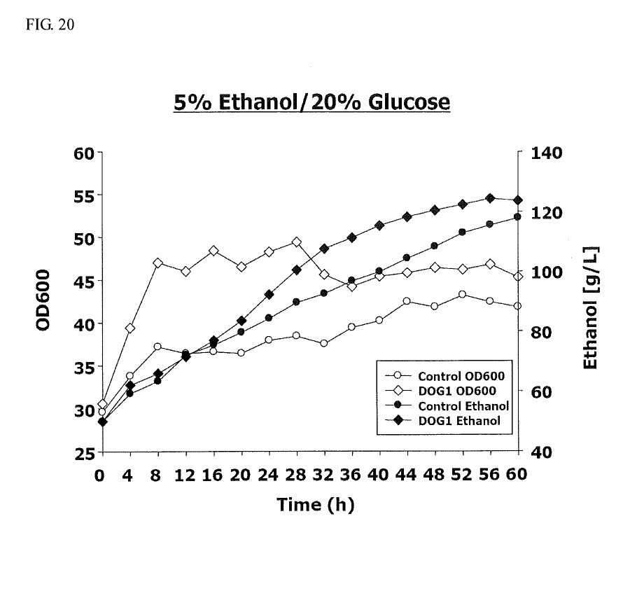

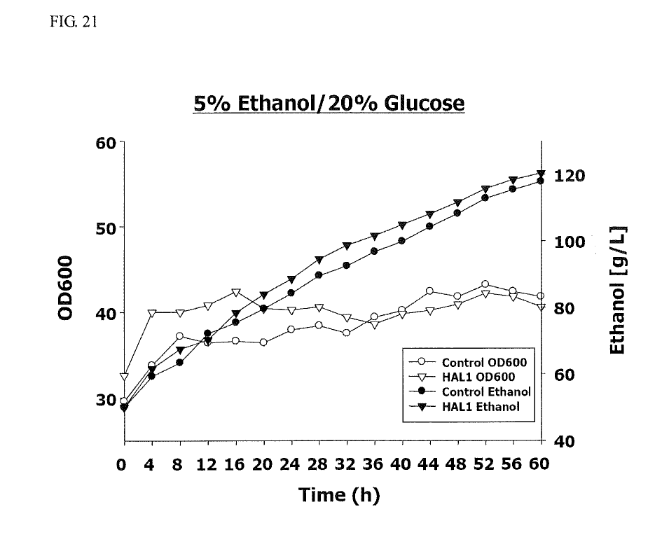

A fermentation test was performed by the same method described in Experimental Example 5 except that the yeast strains of Examples 1 to 4 were fermented in mixed liquid media containing 5% (w/v) ethanol and 20% (w/v) glucose. Viabilities and ethanol volumetric productivities are shown in FIGS. 18 to 21. In addition, specific growth rates (h−1) and ethanol volumetric productivities (g/L/h) are shown in Table 6.

TABLE 6
[5% Ethanol and 20% Glucose Test]

| Parameter | Control | Example 1 | Example 2 | Example 3 | Example 4 |
|---|---|---|---|---|---|
| Specific growth rate(h⁻¹) 0-12 h | 0.017 | 0.030 | 0.032 | 0.034 | 0.019 |
| Volumetric productivity(g/L/h) 0-32 h | 1.294 | 1.427 | 2.025 | 1.858 | 1.457 |

Referring to FIGS. 18 to 21 and Table 6, it can be seen that the strains of Examples 1 to 4 show increases in alcohol tolerance and alcohol volumetric productivity, as compared to the control strain (wild-type). Particularly, it can be seen that the strain of Example 2 shows about a 90% increase in specific growth rate and about a 57% increase in ethanol volumetric productivity, as compared to the control group. It can be also seen that the strain of Example 3 shows about a 100% or more increase in the specific growth rate (0.034), as compared to the control strain (0.017).

### Experimental Example 7

### Fermentation Test according to Concentration Gradient of Glucose

A fermentation test is performed using the yeast strain of Example 2 by the same method described in Experimental Example 5, except that 5% (w/v) ethanol was not contained, and glucose at concentrations of 10%, 20% and 30% (w/v) were used. The fermentation tests were respectively performed at low cell density (OD=about 0.05) and at a high cell density (OD=about 10) under an oxygen-limited condition.

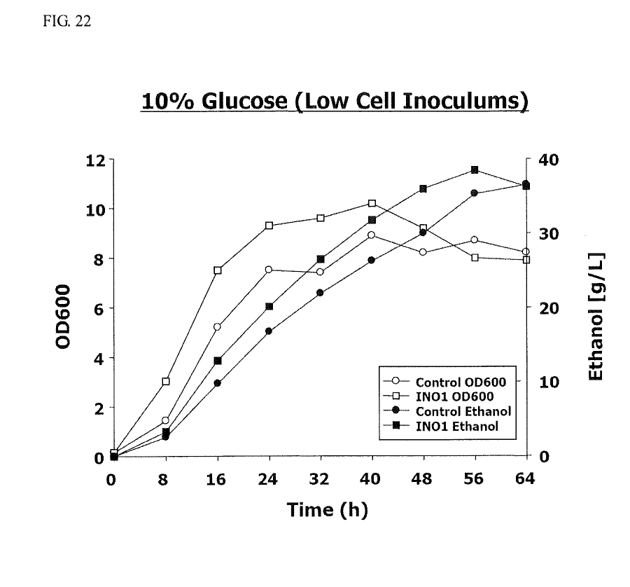

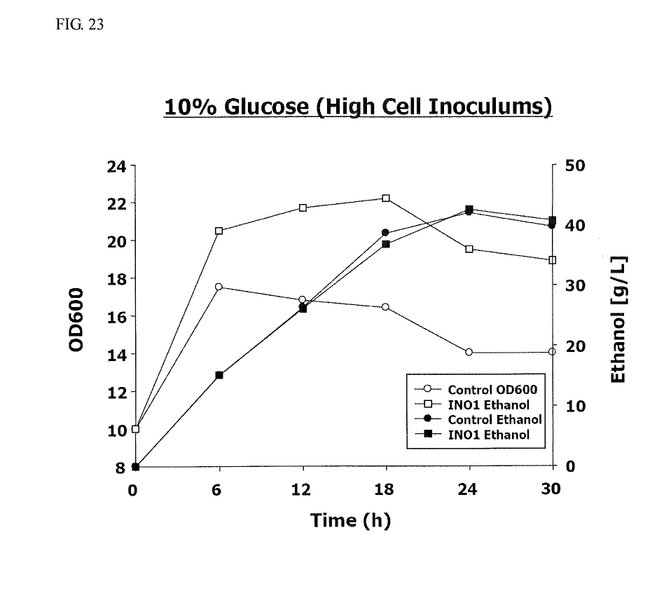

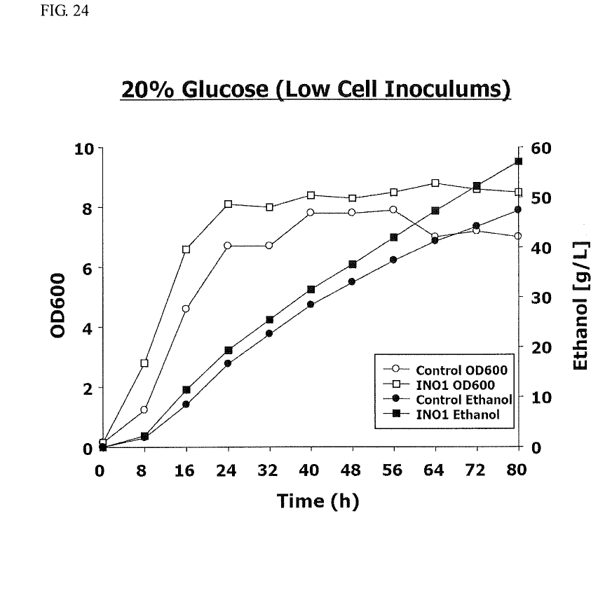

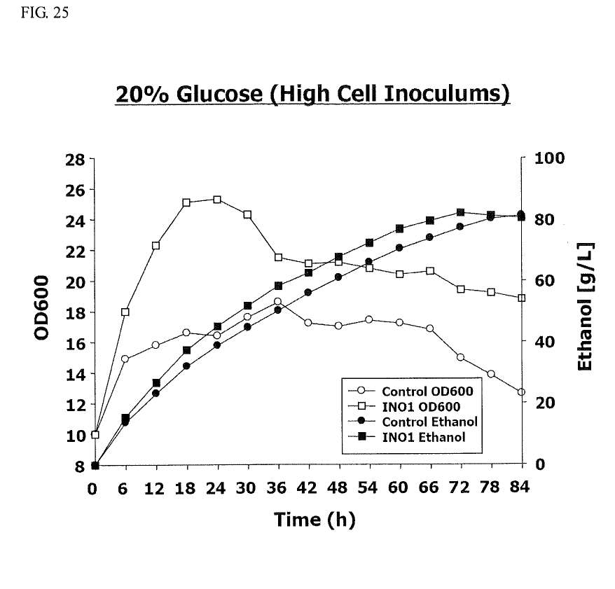

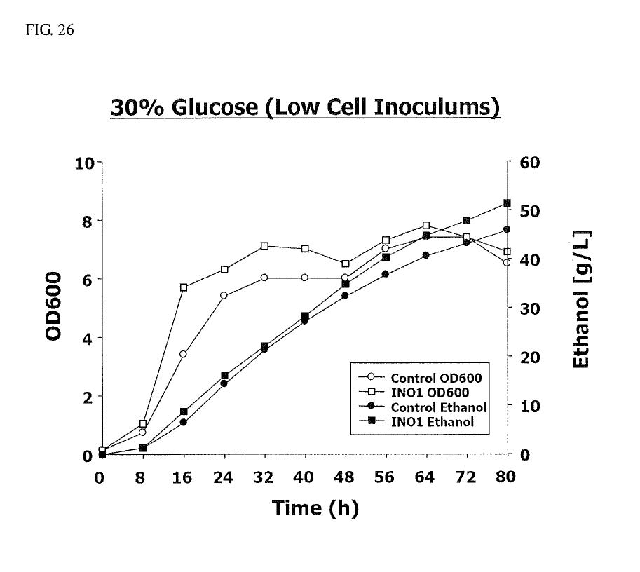

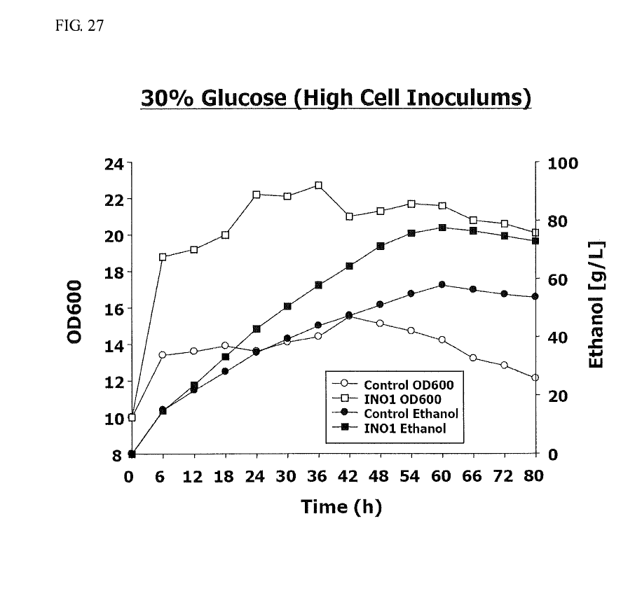

Viabilities and ethanol volumetric productivities are shown in FIGS. 22 to 27. FIGS. 22, 24 and 26 show the results obtained at a low cell density, and FIGS. 23, 25 and 27 show the results obtained at a high cell density. Specific growth rates (h⁻¹) and ethanol volumetric productivities (g/L/h) obtained at low and high cell densities are shown in Tables 7 and 8, respectively.

TABLE 7
[Low Inoculums]

| | 10% Glucose | | 20% Glucose | | 30% Glucose | |
|---|---|---|---|---|---|---|
| Parameter | Control | Example 2 | Control | Example 2 | Control | Example 2 |
| Specific growth rate (h⁻¹) | 0.122 | 0.130 | 0.119 | 0.124 | 0.115 | 0.121 |
| 0 h-32 h | | | | | | |
| Volumetric productivity | 0.625 | 0.750 | 0.685 | 0.761 | 0.694 | 0.725 |
| (g/Lh⁻¹) 0 h-48 h | | | | | | |
| Specific productivity | 0.286 | 0.343 | 0.329 | 0.344 | 0.433 | 0.418 |
| (g/DCWh⁻¹) 0 h-48 h | | | | | | |

TABLE 8
[High Inoculums]

| | 10% Glucose | | 20% Glucose | | 30% Glucose | |
|---|---|---|---|---|---|---|
| Parameter | Control | Example 2 | Control | Example 2 | Control | Example 2 |
| Specific growth rate(h⁻¹) | — | — | 2.701 | 3.135 | 2.514 | 3.004 |
| 0 h-32 h | | | | | | |
| Volumetric productivity | — | — | 1.173 | 1.280 | 0.961 | 1.292 |
| (g/Lh⁻¹) 0 h-48 h | | | | | | |
| Specific productivity | — | — | 0.256 | 0.235 | 0.254 | 0.224 |
| (g/DCWh⁻¹) 0 h-48 h | | | | | | |

Referring to FIGS. 22 to 27 and Tables 7 and 8, it can be seen that once the yeast INO1 gene (set forth in SEQ ID NO: 2) is amplified and expressed, the transformed cell grows more rapidly in glucose and more efficiently converts glucose into ethanol as compared to the control strain. Specifically, it can be seen that in the case of the low cell inoculums, the transformed strain exhibits a higher volumetric productivity than the control parent strain in both 10% glucose and 20% glucose fermentation tests. It can be also seen that in the case of high cell inoculums, the transformed strain shows a higher specific growth rate (0 to 24 h) and higher ethanol volumetric productivity (0 to 60 h) in both 20% and 30% glucose fermentation tests than the control strain.

### Experimental Example 8

### Fermentation Test in Glucose/Galactose Mixed Medium

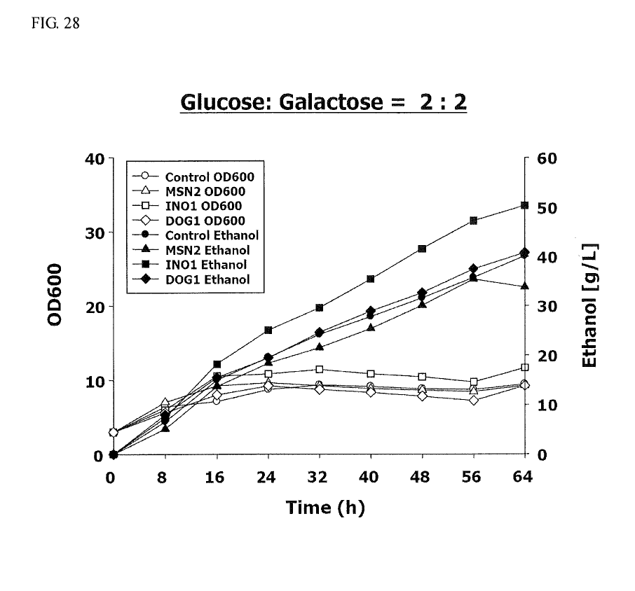

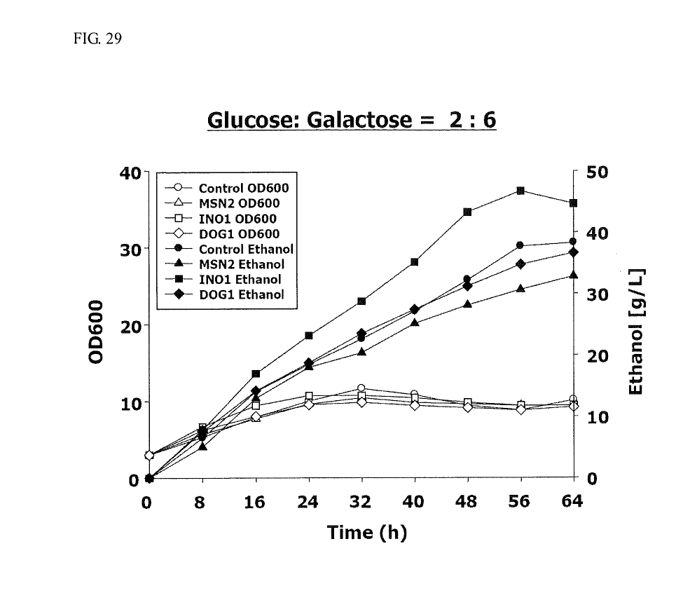

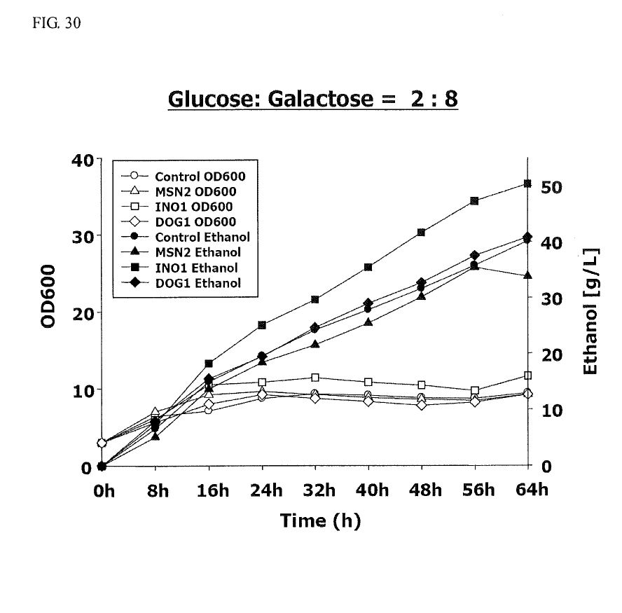

A fermentation test was performed by the same method described in Experimental Example 5, except that the yeast strains of Examples 1 to 3 were fermented in mixed media containing glucose and galactose in various ratios (glucose:galactose=2:2% (w/v), 2:6% (w/v) and 2:8% (w/v)), instead of the mixed media containing 5% (w/v) ethanol and 10% (w/v) glucose. Viability and ethanol volumetric productivity for the yeast strains of Examples 1 to 3 are shown in FIGS. 28 to 30. The ethanol volumetric productivity (g/L/h) is also shown in Table 9 below.

Referring to FIGS. 28 to 30 and Table 9, it can be seen that the strains of Examples 1 to 3 show an increase in alcohol tolerance and alcohol volumetric productivity, as compared to the control strain (wild-type).

TABLE 9

| | Glucose: | | | | |
|---|---|---|---|---|---|
| | Galactose | | | | |
| Parameter | %(w/v) | Control | Example 1 | Example 2 | Example 3 |
| Volumetric | 2:2 | 0.638 | 0.633 | 0.844 | 0.670 |
| productivity | 2:6 | 0.673 | 0.547 | 0.834 | 0.620 |
| (g/Lh⁻¹) | 2:8 | 0.638 | 0.633 | 0.844 | 0.670 |
| 0 h-56 h | | | | | |

### Experimental Example 9

### Viability Test Using Colony Forming Unit (CFU)

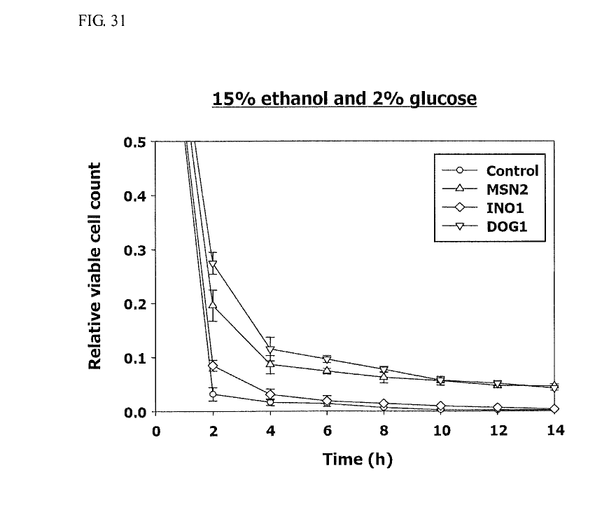

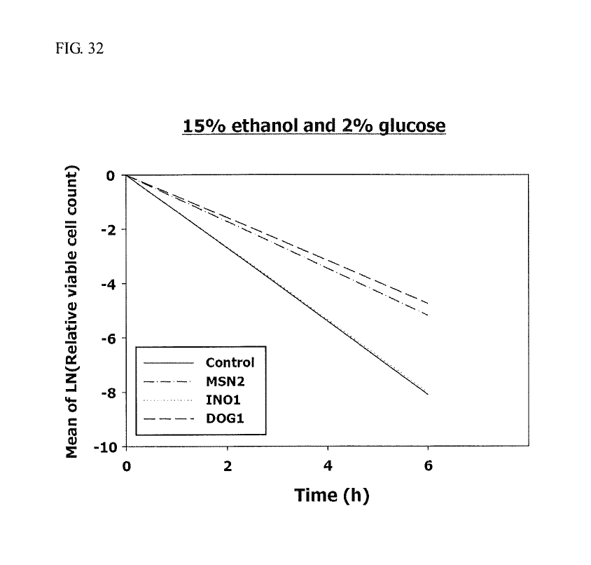

The strains of Examples 1 to 3 were incubated in liquid media containing 15% (w/v) ethanol and 2% (w/v) glucose. After a period of 2, 4, 6, 8, 10 and 12 hours, viability was measured for each strain. The viability is expressed in relative number according to time versus the number of initial colonies, and is shown in FIG. 31. After 2, 4 and 6 hours, the cell death rate was measured, and is shown in FIG. 32. The measurement of the viability and cell death rate was performed according to the method disclosed in “Engineering Yeast Transcription Machinery for Improved Ethanol Tolerance and Production” by Hal Alper et al., published in 8 Dec. 2006, Science 314, 1565 (2006).

Referring to FIGS. 31 and 32, it can be seen that overexpressing strains of Examples 1 to 3 show significant increases in ethanol tolerance, as compared to the control strain (wild-type). Specifically, it can be seen that, in the case of 15% ethanol, the strain of Example 3 (DOG1) shows a 70% increase in ethanol tolerance, and the strains of Examples 1 and 2 also show increases in ethanol tolerance, as compared to the control group.

According to exemplary embodiments, an isolated polynucleotide encodes a protein for enhancing the alcohol tolerance of a host cell, so that the host cell containing the isolated polynucleotide can exhibit excellent viability even in high-concentrations of alcohol, and excellent homeostasis during fermentation. Thus, when the isolated polynucleotide is applied to industrial alcohol fermentation, an enhancement in alcohol volumetric productivity can be achieved, which is very efficient for industrial use.

While exemplary embodiments have been disclosed herein, it should be understood that other variations may be possible. Such variations are not to be regarded as a departure from the spirit and scope of example embodiments of the present application, and all such modifications as would be obvious to one skilled in the art are intended to be included within the scope of the following claims. Moreover, any combination of the above-described elements in all possible variations thereof is encompassed by the invention unless otherwise indicated herein or otherwise clearly contradicted by context.

What is claimed is:

1. An isolated polynucleotide comprising a polynucleotide selected from the group consisting of:
a polynucleotide consisting of a base sequence having at least 90% identity to a base sequence selected from SEQ ID NOs 1 to 8;
a polynucleotide encoding a polypeptide consisting of an amino acid sequence having at least 90% identity to an amino acid sequence selected from SEQ ID NOs 14 to 19;
a polynucleotide consisting of a base sequence which hybridizes to a base sequence selected from SEQ ID NOs: 1 to 8 under stringent conditions; and
a polynucleotide encoding a polypeptide consisting of an amino acid sequence which hybridizes to an amino acid sequence selected from SEQ ID NOs: 14 to 19 under stringent conditions,
wherein the polynucleotide encodes a polypeptide for increasing alcohol tolerance of a host cell.

2. The isolated polynucleotide according to claim 1, wherein the isolated polynucleotide is selected from the group consisting of:
(i) an isolated polynucleotide consisting of base sequence having at least 90% identity to base sequences of SEQ ID NOs: 1 and 9;
(ii) an isolated polynucleotide encoding a polypeptide consisting of amino acid sequences having at least 90% identity to amino acid sequences of SEQ ID NOs: 14 and 21;
(iii) an isolated polynucleotide which hybridizes to the isolated polynucleotide of (i) under stringent conditions; and
(iv) an isolated polynucleotide which hybridizes to the isolated polynucleotide of (ii) under stringent conditions.

3. The isolated polynucleotide according to claim 2, wherein the isolated polynucleotide has a base sequence set forth in SEQ ID NO: 26.

4. The isolated polynucleotide according to claim 1, wherein the isolated polynucleotide is selected from the group consisting of:
(i) an isolated polynucleotide consisting of a base sequence having at least 90% identity to base sequences of SEQ ID NOs: 2, 10 and 11;
(ii) an isolated polynucleotide encoding a polypeptide consisting of an amino acid sequence having at least 90% identity to amino acid sequences of SEQ ID NOs: 15, 22 and 23;
(iii) an isolated polynucleotide which hybridizes to the isolated polynucleotide of (i) under stringent conditions; and
(iv) an isolated polynucleotide which hybridizes to the isolated polynucleotide of (ii) under stringent conditions.

5. The isolated polynucleotide according to claim 4, wherein the isolated polynucleotide has a base sequence set forth in SEQ ID NO: 27.

6. The isolated polynucleotide according to claim 1, wherein the isolated polynucleotide is a polynucleotide derived from Saccharomyces cerevisiae (S. cerevisiae).

7. The isolated polynucleotide according to claim 1, wherein the alcohol tolerance is expressed as a specific cell growth rate (h⁻¹) in a minimum inhibition concentration (MIC).

8. The isolated polynucleotide according to claim 7, wherein the MIC is about 5% for ethanol or about 1% for isobutanol.

9. The isolated polynucleotide according to claim 1, wherein the isolated polynucleotide is selected from the group consisting of:
an isolated polynucleotide consisting of a base sequence having at least 90% identity to a base sequence selected from SEQ ID NOs: 28 to 31; and
an isolated polynucleotide consisting of a base sequence which hybridizes to a base sequence selected from SEQ ID NOs: 28 to 31 under stringent conditions.

10. A vector comprising an isolated polynucleotide according to claim 1.

11. The vector according to claim 10, wherein the vector is a plasmid.

12. A host cell capable of producing alcohol when incubated in a monosaccharide-containing nutrient source, and which exhibits overexpression of one or more isolated polynucleotides encoding a polypeptide for increasing alcohol tolerance of the host cell, wherein the isolated polynucleotide is selected from the group consisting of:
a polynucleotide consisting of a base sequence having at least 90% identity to a base sequence selected from SEQ ID NOs: 1 to 8;
a polynucleotide encoding a polypeptide consisting of an amino acid sequence having at least 90% identity to an amino acid sequence selected from SEQ ID NOs: 14 to 19;
a polynucleotide consisting of a base sequence which hybridizes to a base sequence selected from SEQ ID NOs: 1 to 8 under stringent conditions; and
a polynucleotide encoding a polypeptide consisting of an amino acid sequence which hybridizes to an amino acid sequence selected from SEQ ID NOs: 14 to 19 under stringent conditions.

13. The host cell according to claim 12, wherein the host cell is a species of the genus Saccharomyces.

14. The host cell according to claim 12, wherein the monosaccharide is glucose, galactose or a combination thereof.

15. The host cell according to claim 12, wherein the host cells exhibits at least 30% increase in specific growth rate (h⁻¹) in minimum inhibition concentration (MIC), as compared to wild-type S. cerevisiae.

16. The host cell according to claim 12, wherein the MIC is about 5% for ethanol or about 1% for isobutanol.

17. The host cell according to claim 12, wherein the host cell exhibits at least a 10% increase in volumetric productivity of ethanol (g/L/h) as compared to wild-type Saccharomyces cerevisiae (S. cerevisiae) under the same incubation conditions.

18. The host cell according to claim 12, wherein the host cell exhibits overexpression of a polynucleotide consisting of a base sequence having at least 90% identity to a base sequence of SEQ ID NO: 26 or 27.

19. The host cell according to claim 12, wherein the host cell is selected from the group consisting of:
a host cell derived from S. cerevisiae CEN.PK2-1D/pRS424-MSN2/MIH1 deposited with the Genebank of the Korea Research Institute of Bioscience and Biotechnology under Accession No. KCTC11476BP;
a host cell derived from S. cerevisiae CEN.PK2-1D/pRS424-INO1 deposited with the Genebank of the Korea Research Institute of Bioscience and Biotechnology under Accession No. KCTC11477BP;
a host cell derived from S. cerevisiae CEN.PK2-1D/pRS424-DOG1 deposited with the Genebank of the Korea Research Institute of Bioscience and Biotechnology under Accession No. KCTC11478BP;
a host cell derived from S. cerevisiae CEN.PK2-1D/pRS424-HAL1 deposited with the Genebank of the Korea Research Institute of Bioscience and Biotechnology under Accession No. KCTC11479BP
a host cell derived from S. cerevisiae CEN.PK2-1D/pRS424-TRP1 deposited with the Genebank of the Korea Research Institute of Bioscience and Biotechnology under Accession No. KCTC11480BP
a host cell derived from S. cerevisiae CEN.PK2-1D/pRS424-MRPL17 deposited with the Genebank of the Korea Research Institute of Bioscience and Biotechnology under Accession No. KCTC11481BP;
a host cell derived from S. cerevisiae CEN.PK2-1D/pRS424-YLR157C-B deposited with the Genebank of the Korea Research Institute of Bioscience and Biotechnology under Accession No. KCTC11482BP; and
a host cell derived from S. cerevisiae CEN.PK2-1D/pRS424-SPG5p deposited with the Genebank of the Korea Research Institute of Bioscience and Biotechnology under Accession No. KCTC11483BP.

20. The host cell according to claim 12, wherein the overexpression is achieved by increasing the number of copies of the polynucleotide.

21. A method of producing bioalcohol comprising incubating the host cell according to claim 12 in a monosaccharide-containing nutrient source and producing alcohol through fermentation.

22. The method according to claim 21, further comprising:
engineering a host cell to overexpress one or more isolated polynucleotides encoding a polypeptide for increasing alcohol tolerance of the host cell, wherein the isolated polynucleotide is at least one selected from the group consisting of: (a) a polynucleotide consisting of a base sequence having at least 90% identity to a base sequence selected from SEQ ID NOs: 1 to 8, (b) a polynucleotide encoding a polypeptide consisting of an amino acid sequence having at least 90% identity to an amino acid sequence selected from SEQ ID NOs: 14 to 19, (c) a polynucleotide consisting of a base sequence which hybridizes to a base sequence selected from SEQ ID NOs: 1 to 8 under stringent conditions, and (d) a polynucleotide encoding a polypeptide consisting of an amino acid sequence which hybridizes to an amino acid sequence selected from SEQ ID NOs: 14 to 19 under stringent conditions; and
incubating the host cell in a monosaccharide-containing nutrient source under conditions suitable for producing alcohol; and
producing alcohol through fermentation.

23. The method according to claim 22, wherein the engineering of the host cell includes inserting the isolated polynucleotide encoding a polypeptide into a vector, amplifying the vector, and inserting the vector into the host cell.

24. The method according to claim 21, wherein the host cell is a yeast cell.

25. The method according to claim 21, wherein the incubating of the host cell is performed by stirring at a rate of about 100 to about 250 rpm at an initial glucose concentration of about 2 to about 30% (w/v), a temperature of about 25 to about 37° C., and a pH of about 5.0 to about 8.0.


SEQUENCE LISTING (31 sequences)

SEQ ID NO: 1 - DNA, 377 bp, Artificial Sequence
  trucated MIH1
atgaacaatatatttcatggaactgaagatgaatgtgccaatgaagacgttcttagtttccaaaaaatttccttgaaaagtccctttggtaagaagaagaacatatttagaaatgttcagaccttctttaagtcaaaaagcaaacattcgaatgtcgacgatgatttaatcaataaagagaatcttgcctttgataaatctccattgttaacaaatcacaggagtaaggaaattgatggtccttcaccgaatataaagcagcttggccatcgcgatgagttagatgaaaatgaaaatgaaaatgatgatatagtcttaagcatgcattttgcttctcaaaccttacaaagtccaacaagaaactcatcaagaagatc

SEQ ID NO: 2 - DNA, 1602 bp, Artificial Sequence
  INO1
atgacagaagataatattgctccaatcacctccgttaaagtagttaccgacaagtgcacgtacaaggacaacgagctgctcaccaagtacagctacgaaaatgctgtagttacgaagacagctagtggccgcttcgatgtaacgcccactgttcaagactacgtgttcaaacttgacttgaaaaagccggaaaaactaggaattatgctcattgggttaggtggcaacaatggctccactttagtggcctcggtattggcgaataagcacaatgtggagtttcaaactaaggaaggcgttaagcaaccaaactacttcggctccatgactcaatgttctaccttgaaactgggtatcgatgcggaggggaatgacgtttatgctccttttaactctctgttgcccatggttagcccaaacgactttgtcgtctctggttgggacatcaataacgcagatctatacgaagctatgcagagaagtcaagttctcgaatatgatctgcaacaacgcttgaaggcgaagatgtccttggtgaagcctcttccttccatttactaccctgatttcattgcagctaatcaagatgagagagccaataactgcatcaatttggatgaaaaaggcaacgtaaccacgaggggtaagtggacccatctgcaacgcatcagacgcgatatccagaatttcaaagaagaaaacgcccttgataaagtaatcgttctttggactgcaaatactgagaggtacgtagaagtatctcctggtgttaatgacaccatggaaaacctcttgcagtctattaagaatgaccatgaagagattgctccttccacgatctttgcagcagcatctatcttggaaggtgtcccctatattaatggttcaccgcagaatacttttgttcccggcttggttcagctggctgagcatgagggtacattcattgcgggagacgatctcaagtcgggacaaaccaagttgaagtctgttctggcccagttcttagtggatgcaggtattaaaccggtctccattgcatcctataaccatttaggcaataatgacggttataacttatctgctccaaaacaatttaggtctaaggagatttccaaaagttctgtcatagatgacatcatcgcgtctaatgatatcttgtacaatgataaactgggtaaaaaagttgaccactgcattgtcatcaaatatatgaagcccgtcggggactcaaaagtggcaatggacgagtattacagtgagttgatgttaggtggccataaccggatttccattcacaatgtttgcgaagattctttactggctacgcccttgatcatcgatcttttagtcatgactgagttttgtacaagagtgtcctataagaaggtggacccagttaaagaagatgctggcaaattcgagaacttttatccagttttaaccttcttgagttactggttaaaagctccattaacaagaccaggatttcacccggtgaatggcttaaacaagcaaagaaccgccttagaaaattttttaagattgttgattggattgccttctcaaaacgaactaagattcgaagagagattgttgtaa

SEQ ID NO: 3 - DNA, 741 bp, Artificial Sequence
  DOG1
atggcagaattttcagctgatctatgtctttttgacctagatggtaccatagtgagtacaacagtggccgcagagaaagcatggaccaagttgtgttacgaatacggtgttgatccttccgagttatttaagcattctcatggtgcaagaacacaagaggttttgagaaggtttttccctaaattggatgatacagacaataaaggtgttcttgctctagaaaaagatattgcccatagttacttggacacagtaagccttattcctggtgcagagaacttactgttatcgttagatgtagatactgagactcaaaaaaagttacctgaaaggaaatgggctatcgttacctctggttctccatatttggcattttcatggttcgagacaatattgaaaaatgttggaaagcccaaagttttcattactgggtttgacgtgaagaacggtaagcctgatcccgagggttattcaagagctcgtgatttattgcgtcaagatttgcaattaactggtaaacaggatctgaagtatgttgtcttcgaagatgcacccgtgggcataaaggccggcaaagcaatgggcgccattactgtgggtataacatcctcgtatgacaagagcgttttatttgacgcaggagcagattatgtagtctgtgatttgacacaggtttccgtggttaagaacaatgaaaacggtattgtcatccaggtaaacaaccctttgacaagggcctga

SEQ ID NO: 4 - DNA, 885 bp, Artificial Sequence
  HAL1
atgcatttcaaagatttaggattgcatgactacactctcaaaaacttgatgtatgagaataattgctgtaaattttatgatgccgtggatgaaaacaacatctcatatgttttaaaatttgttccctcagatgtgacttcggaaggggatactttcccattcgtggatcgctttcaagtaaaggaaggtgtttttttggtatattcctcaaatgactttggaaaagaaggtacggactactttacttatactggtagtggtggaaatgaggttcacatctcgggcacctcttcagaagcaggaataaaaccgcagtttattgaaacttgccatccaaaacatcttaagcggggaacaaaagagcaggaagatataaatagtagtacctcaaagaaaagtgcagttatcaacaatttttcgggtgaaaaaacaccaaatccaaggccacagagttccaacatttcagaaagagagacgtatgtcggaatattgaacgtcaaatgtaaaaataagaactcatcgaaaatacgaagtgaaaaattggtaagctccgtcatcgaaacaaagcatacgccaggattggcatctattttatcgaaagaaggcactacatatccgaataatgcggacgggaaacatatcagtatcgtgaatccatcctcaaaaatatatcattcatcccataaacagattgttaaaacgcctatccctaagagtggcctttctccaattgagagatgccctttcaatggtcaaaatattaaatgctactcaccaagaccactagatcatgaaagtccccaacgtgatttcaataataactttcagctgagaatactgaagagctcggtgttgcaaaggagacaatcaacacagaatagttga

SEQ ID NO: 5 - DNA, 668 bp, Artificial Sequence
  TRP1
atgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaa

SEQ ID NO: 6 - DNA, 790 bp, Artificial Sequence
  truncated MRPL17
atgaaggtaaatttaatgttgaaaagagggcttgctactgcaactgcaactgccagttccgctccccccaagattaaagtcggagtactactgtcaagaatccctataattaaatcagaattaaatgaactagagaaaaaatactatgagtaccaatcagaactagaaaagagactaatgtggacgtttccggcatatttttatttcaaaaagggtactgtagcagaacacaaatttctatccctgcagaaaggacctatctccaaaaaaaatggcatttggtttcctagaggcataccggacattaaacatggcagagaaagaagtactaagcaagaagttaaactttctgatgacagtacagtagcatttagcaacaatcaaaaagagcaaagcaaagacgatgttaataggcccgtgattcccaacgacaggataacggaagcagataggtcaaatgatatgaagagccttgaaagacaattgagcaggaccttatatcttttggttaaggataaaagcggtacttggaaattccctaacttcgatctttctgatgaatctaagccgttacacgtacacgcagagaacgaattgaaattgttgagcggtgatcagatatacacttggtctgtttctgctacgcccataggtgttttgcaggacgagagaaataggactgctgagtttattgtgaagtcacacattttggctggaaaatttgatttggtggcgtcgaaaaatgatgcattcgaggattttgcttggctgacaaaaggtgagatcagtg

SEQ ID NO: 7 - DNA, 1468 bp, Artificial Sequence
  Partial fragment YLR157C-B
attacaggagaaatctgagtgatgagaagaatgattctcgcagctatacgaatacaaccaaacccaaagttatagctcggaatcctcaaaaaacaaataattcgaaatcgaaaacagccagggctcacaatgtatccacatctaataactctcccagcacggacaacgattccatcagtaaatcaactactgaaccgattcaattgaacaataagcacgaccttcaccttaggccaggaacttactgaatctacggtaaatcacactaatcattctgatgatgaactccctggacacctccttctcgattcaggagcatcacgaacccttataagatctgctcatcacatacactcagcatcatctaatcctgacataaacgtagttgatgctcaaaaaagaaatataccaattaacgctattggtgacctacaatttcacttccaggacaacaccaaaacatcaataaaggtattgcacactcctaacatagcctatgacttactcagtttgaatgaattggctgcagtagatatcacagcatgctttaccaaaaacgtcttagaacgatctgacggcactgtacttgcacctatcgtaaaatatggagacttttactgggtatctaaaaagtacttgcttccatcaaatatctccgtacccaccatcaataatgtccatacaagtgaaagtacacgcaaatatccttatcctttcattcatcgaatgcttgcgcatgccaatgcaccgacaattcgatactcacttaaaaataacaccatcacgtattttaacgaatcagatgtcgactggtctagtgctattgactatcaatgtcctgattgtttaatcggcaaaagcaccaaacacagacatatcaaaggttcacgactaaaataccaaaattcatacgaaccctttcaatacctacatactgacatatttggtccagttcacaacctaccaaatagtgcaccatcctatttcatctcatttactgatgagacaacaaaattccgttgggtttatccattacacgaccgtcgcgaggactctatcctcgatgtttttactacgatactagcttttattaagaaccagtttcaggccagtgtcttggttatacaaatggaccgtggttctgagtatactaacagaactctccataaattccttgaaaaaaatggtataactccatgctatacaaccacagcggattcccgagcacatggagtcgctgaacggctcaaccgtaccttattagatgactgccgtactcaactgcaatgtagtggtttaccgaaccatttatggttctctgcaatcgaattttctactattgtgagaaattcactagcttcacctaaaagcaaaaaatctgcaagacaacatgctggcttggcaggacttgatatcagtactttgttacctttcggtcaacctgttatcgtcaatgatcacaacccta

SEQ ID NO: 8 - DNA, 179 bp, Artificial Sequence
  Putative SPG5 promoter
ccgtatatcagcttttagatcaggctcgagtttcttgttatatgtgcattgcaaaagcataaacaaatcctggcagccgaagccgggcaatccacttcgaaacgcacggctgaactatataaatataaaggacatgtggagagaagcttctcttccttcacatttcgcatttcatgatc

SEQ ID NO: 9 - DNA, 1943 bp, Artificial Sequence
  trucated MSN2
aaaatcaagaaacttcactgaatttggggcttcctccactatctttcgactctccactgcccgtaacggaaacgataccatccactaccgataacagcttgcatttgaaagctgatagcaacaaaaatcgcgatgcaagaactattgaaaatgatagtgaaattaagagtactaataatgctagtggctctggggcaaatcaatacacaactcttacttcaccttatcctatgaacgacattttgtacaacatgaacaatccgttacaatcaccgtcaccttcatcggtacctcaaaatccgactataaatcctcccataaatacagcaagtaacgaaactaatttatcgcctcaaacttcaaatggtaatgaaactcttatatctcctcgagcccaacaacatacgtccattaaagataatcgtctgtccttacctaatggtgctaattcgaatcttttcattgacactaacccaaacaatttgaacgaaaaactaagaaatcaattgaactcagatacaaattcatattctaactccatttctaattcaaactccaattctacgggtaatttaaattccagttattttaattcactgaacatagactccatgctagatgattacgtttctagtgatctcttattgaatgatgatgatgatgacactaatttatcacgccgaagatttagcgacgttataacaaaccaatttccgtcaatgacaaattcgaggaattctatttctcactctttggacctttggaaccatccgaaaattaatccaagcaatagaaatacaaatctcaatatcactactaattctacctcaagttccaatgcaagtccgaataccactactatgaacgcaaatgcagactcaaatattgctggcaacccgaaaaacaatgacgctaccatagacaatgagttgacacagattcttaacgaatataatatgaacttcaacgataatttgggcacatccacttctggcaagaacaaatctgcttgcccaagttcttttgatgccaatgctatgacaaagataaatccaagtcagcaattacagcaacagctaaaccgagttcaacacaagcagctcacctcgtcacataataacagtagcactaacatgaaatccttcaacagcgatctttattcaagaaggcaaagagcttctttacccataatcgatgattcactaagctacgacctggttaataagcaggatgaagaccccaagaacgatatgctgccgaattcaaatttgagttcatctcaacaatttatcaaaccgtctatgattctttcagacaatgcgtccgttattgcgaaagtggcgactacaggcttgagtaatgatatgccatttttgacagaggaaggtgaacaaaatgctaattctactccaaatttcgatctttccatcactcaaatgaatatggctccattatcgcctgcatcatcatcctccacgtctcttgcaacaaatcatttctatcaccatttcccacagcagggtcaccataccatgaactctaaaatcggttcttcccttcggaggcggaagtctgctgtgcctttgatgggtacggtgccgcttacaaatcaacaaaataatataagcagtagtagtgtcaactcaactggcaatggtgctggggttacgaaggaaagaaggccaagttacaggagaaaatcaatgacaccgtccagaagatcaagtgtcgtaatagaatcaacaaaggaactcgaggagaaaccgttccactgtcacatttgtcccaagagctttaagcgcagcgaacatttgaaaaggcatgtgagatctgttcactctaacgaacgaccatttgcttgtcacatatgcgataagaaatttagtagaagcgataatttgtcgcaacacatcaagactcataaaaaacatggagacatttaa

SEQ ID NO: 10 - DNA, 855 bp, Artificial Sequence
  truncated VPS35
atggcgtatgcggactcaccagaaaatgcgatcgctgttatcaagcagcgaaccgcactaatgaaccggtgtctatctcaacacaaactaatggaatcattacagcatacttccataatgttgaccgaattgagaaatccaaacttatcgccgaagaaatactacgaactttatgtcattattttcgactcattgactaatctatctacgtacctcatagaaaaccatcctcaaaatcaccacttagctgatctttatgagttggttcaatataccggtaacgtggtacccaggctttacttgatgatcacagttgggaccagctatctcactttcaatgaagcgcccaagaaggaaatcttaaaggatatgattgagatgtgtcgtggtgtgcaaaacccaataagaggtttgtttttacgctattatttatcccagagaaccaaagaattacttccggaggacgatccgtcgtttaactctcaatttattatgaataatttcatcgagatgaacaagttgtgggtgagattacaacaccaggggccacttcgtgagagggaaaccagaacacgtgaaagaaaagagctgcaaattttagtggggtctcaactagtacgtctttcgcagattattgatgataatttccaaatgtataagcaagatattcttcccaccattttggaacaagtcatacaatgtagagatttagtatcccaagaatatcttttggacgtcatctgccaagtgttcgcagacgagttccatttgaaaaccttggatactttactgcaaactactttgcatttgaaccctgatgtttcgataaacaagattgttctcactttggtcgat

SEQ ID NO: 11 - DNA, 168 bp, Artificial Sequence
  truncated SNA3
cgatctttcacgcagacatgcgactgcgcccgccgtagaccgtgacctggaagctcaccctgcagaggaatctcaagcacagcctccagcatatgatgaagacgatgaggccggtgccgatgtgcccttgatggacaacaaacaacagctctcttccggccgtactta

SEQ ID NO: 12 - DNA, 636 bp, Artificial Sequence
  truncated YHR045W
tgattcgataggatcatcgcaaacaacaataagcttgtaccaatcctcgctgccgtttagcatcttaaaagacttccatgagtcgatgatcaggacatcaacatcttctagtcgttgtctaggaacagctggcaagaaatgaggtattgaacctgttcttatagaggtcatcatggaagccaaggagagagtaaaaccctctacggtagcaatagaattagctataccgatatttttgaaattcttattggaaagtttcggaaatatgcgcttggccataccattaagttcactcaacgaataacttttttctcttcctgtaaatttaattatgttttttcccttggaaacttccattatggcattccaaacatcaccgaaattgccatttcttattttgtattttaaagatagccctaatcctgtcgtcaatgggaatcctgtgggcactaaaatgcttcgataatatgctgtttcattttctttacgaacactggatatgctagattgctgtgacaaagccacccctgttaaatctctcttaaaatcaagtagaaaagagcttagcaaccagttggcgcccagcacaattaacaatattgttattaccagctgtagtagaaaagaccaattcat

SEQ ID NO: 13 - DNA, 938 bp, Artificial Sequence
  truncated AIM45
atgtttaaatcattggctgctgtcttgcctagagctagcaaggcaaagttcctccagaaaaattacgcctccactttagctttcattgaaagctcaaaagatggctctgtttcaaggtcatcattgagtttattggctgctgcacaaaagttgtctaaccctatcacagctgtaatcacaggtagcaaagctgaaaaaactgctgaggcgctaaaatcttcatattcatgcagcaatttagaaaagcttgtcatatttgaagattcaaaattagatacctgtcttcccgaacaactaactccgttattagtgaaactattaaaaggcggcgactattcacattttgttgtctcaaactcctctgttggaaaaagtgttttacctcgggtgggtgcgctcttggacgtccaacctgtttgtgaggttactgtaatcaaagatcctaagacctttataaggccaatttatgcaggtaacattatttctacaatagaatgccaggcagaaaaaaaactgttgattattagggcatcagcttttccaccaattgcagagggtagtatggattctgttaccattgagaagagaactgatattcctccttgtgacttaaatgttacctgggttaaaactattcttaccaagagtgaaaggcctgaacttacttctgcacagaacgtggtaactggtggaagggcactcaaggataaggagacatttgagaagctattatcgccgctagcagatgttttgcacgctgctataggtgccacaagagcttctgttgataatggactatgtgataattctctacaaatcggtcagactggtaaggtagtcgcaccaaatttgtatatagccattggcgtttctggtgcagttcagcatttagcgggaatgaaggattcgaaagttatcgttgccattaacaatgatcc

SEQ ID NO: 14 - protein, 125 aa, Artificial Sequence
  trucated MIH1
MNNIFHGTEDECANEDVLSFQKISLKSPFGKKKNIFRNVQTFFKSKSKHSNVDDDLINKENLAFDKSPLLTNHRSKEIDGPSPNIKQLGHRDELDENENENDDIVLSMHFASQTLQSPTRNSSRR

SEQ ID NO: 15 - protein, 533 aa, Artificial Sequence
  INO1
MTEDNIAPITSVKVVTDKCTYKDNELLTKYSYENAVVTKTASGRFDVTPTVQDYVFKLDLKKPEKLGIMLIGLGGNNGSTLVASVLANKHNVEFQTKEGVKQPNYFGSMTQCSTLKLGIDAEGNDVYAPFNSLLPMVSPNDFVVSGWDINNADLYEAMQRSQVLEYDLQQRLKAKMSLVKPLPSIYYPDFIAANQDERANNCINLDEKGNVTTRGKWTHLQRIRRDIQNFKEENALDKVIVLWTANTERYVEVSPGVNDTMENLLQSIKNDHEEIAPSTIFAAASILEGVPYINGSPQNTFVPGLVQLAEHEGTFIAGDDLKSGQTKLKSVLAQFLVDAGIKPVSIASYNHLGNNDGYNLSAPKQFRSKEISKSSVIDDIIASNDILYNDKLGKKVDHCIVIKYMKPVGDSKVAMDEYYSELMLGGHNRISIHNVCEDSLLATPLIIDLLVMTEFCTRVSYKKVDPVKEDAGKFENFYPVLTFLSYWLKAPLTRPGFHPVNGLNKQRTALENFLRLLIGLPSQNELRFEERLL

SEQ ID NO: 16 - protein, 246 aa, Artificial Sequence
  DOG1
MAEFSADLCLFDLDGTIVSTTVAAEKAWTKLCYEYGVDPSELFKHSHGARTQEVLRRFFPKLDDTDNKGVLALEKDIAHSYLDTVSLIPGAENLLLSLDVDTETQKKLPERKWAIVTSGSPYLAFSWFETILKNVGKPKVFITGFDVKNGKPDPEGYSRARDLLRQDLQLTGKQDLKYVVFEDAPVGIKAGKAMGAITVGITSSYDKSVLFDAGADYVVCDLTQVSVVKNNENGIVIQVNNPLTRA

SEQ ID NO: 17 - protein, 294 aa, Artificial Sequence
  HAL1
MHFKDLGLHDYTLKNLMYENNCCKFYDAVDENNISYVLKFVPSDVTSEGDTFPFVDRFQVKEGVFLVYSSNDFGKEGTDYFTYTGSGGNEVHISGTSSEAGIKPQFIETCHPKHLKRGTKEQEDINSSTSKKSAVINNFSGEKTPNPRPQSSNISERETYVGILNVKCKNKNSSKIRSEKLVSSVIETKHTPGLASILSKEGTTYPNNADGKHISIVNPSSKIYHSSHKQIVKTPIPKSGLSPIERCPFNGQNIKCYSPRPLDHESPQRDFNNNFQLRILKSSVLQRRQSTQNS

SEQ ID NO: 18 - protein, 224 aa, Artificial Sequence
  TRP1
MSVINFTGSSGPLVKVCGLQSTEAAECALDSDADLLGIICVPNRKRTIDPVIARKISSLVKAYKNSSGTPKYLVGVFRNQPKEDVLALVNDYGIDIVQLHGDESWQEYQEFLGLPVIKRLVFPKDCNILLSAASQKPHSFIPLFDSEAGGTGELLDWNSISDWVGRQESPESLHFMLAGGLTPENVGDALRLNGVIGVDVSGGVETNGVKDSNKIANFVKNAKK

SEQ ID NO: 19 - protein, 262 aa, Artificial Sequence
  truncated MRPL17
MKVNLMLKRGLATATATASSAPPKIKVGVLLSRIPIIKSELNELEKKYYEYQSELEKRLMWTFPAYFYFKKGTVAEHKFLSLQKGPISKKNGIWFPRGIPDIKHGRERSTKQEVKLSDDSTVAFSNNQKEQSKDDVNRPVIPNDRITEADRSNDMKSLERQLSRTLYLLVKDKSGTWKFPNFDLSDESKPLHVHAENELKLLSGDQIYTWSVSATPIGVLQDERNRTAEFIVKSHILAGKFDLVASKNDAFEDFAWLTKGEI

SEQ ID NO: 20 - skipped

SEQ ID NO: 21 - protein, 646 aa, Artificial Sequence
  trucated MSN2
NQETSLNLGLPPLSFDSPLPVTETIPSTTDNSLHLKADSNKNRDARTIENDSEIKSTNNASGSGANQYTTLTSPYPMNDILYNMNNPLQSPSPSSVPQNPTINPPINTASNETNLSPQTSNGNETLISPRAQQHTSIKDNRLSLPNGANSNLFIDTNPNNLNEKLRNQLNSDTNSYSNSISNSNSNSTGNLNSSYFNSLNIDSMLDDYVSSDLLLNDDDDDTNLSRRRFSDVITNQFPSMTNSRNSISHSLDLWNHPKINPSNRNTNLNITTNSTSSSNASPNTTTMNANADSNIAGNPKNNDATIDNELTQILNEYNMNFNDNLGTSTSGKNKSACPSSFDANAMTKINPSQQLQQQLNRVQHKQLTSSHNNSSTNMKSFNSDLYSRRQRASLPIIDDSLSYDLVNKQDEDPKNDMLPNSNLSSSQQFIKPSMILSDNASVIAKVATTGLSNDMPFLTEEGEQNANSTPNFDLSITQMNMAPLSPASSSSTSLATNHFYHHFPQQGHHTMNSKIGSSLRRRKSAVPLMGTVPLTNQQNNISSSSVNSTGNGAGVTKERRPSYRRKSMTPSRRSSVVIESTKELEEKPFHCHICPKSFKRSEHLKRHVRSVHSNERPFACHICDKKFSRSDNLSQHIKTHKKHGDI

SEQ ID NO: 22 - protein, 284 aa, Artificial Sequence
  Truncated VPS35
MAYADSPENAIAVIKQRTALMNRCLSQHKLMESLQHTSIMLTELRNPNLSPKKYYELYVIIFDSLTNLSTYLIENHPQNHHLADLYELVQYTGNVVPRLYLMITVGTSYLTFNEAPKKEILKDMIEMCRGVQNPIRGLFLRYYLSQRTKELLPEDDPSFNSQFIMNNFIEMNKLWVRLQHQGPLRERETRTRERKELQILVGSQLVRLSQIIDDNFQMYKQDILPTILEQVIQCRDLVSQEYLLDVICQVFADEFHLKTLDTLLQTTLHLNPDVSINKIVLTLV

SEQ ID NO: 23 - protein, 55 aa, Artificial Sequence
  Truncated SNA3
DLSRRHATAPAVDRDLEAHPAEESQAQPPAYDEDDEAGADVPLMDNKQQLSSGRT

SEQ ID NO: 24 - protein, 212 aa, Artificial Sequence
  Truncated YHR045W
MNWSFLLQLVITILLIVLGANWLLSSFLLDFKRDLTGVALSQQSSISSVRKENETAYYRSILVPTGFPLTTGLGLSLKYKIRNGNFGDVWNAIMEVSKGKNIIKFTGREKSYSLSELNGMAKRIFPKLSNKNFKNIGIANSIATVEGFTLSLASMMTSIRTGSIPHFLPAVPRQRLEDVDVLIIDSWKSFKMLNGSEDWYKLIVVCDDPIES

SEQ ID NO: 25 - protein, 312 aa, Artificial Sequence
  truncated AIM45
MFKSLAAVLPRASKAKFLQKNYASTLAFIESSKDGSVSRSSLSLLAAAQKLSNPITAVITGSKAEKTAEALKSSYSCSNLEKLVIFEDSKLDTCLPEQLTPLLVKLLKGGDYSHFVVSNSSVGKSVLPRVGALLDVQPVCEVTVIKDPKTFIRPIYAGNIISTIECQAEKKLLIIRASAFPPIAEGSMDSVTIEKRTDIPPCDLNVTWVKTILTKSERPELTSAQNVVTGGRALKDKETFEKLLSPLADVLHAAIGATRASVDNGLCDNSLQIGQTGKVVAPNLYIAIGVSGAVQHLAGMKDSKVIVAINND

SEQ ID NO: 26 - DNA, 3202 bp, Artificial Sequence
  trucated MSN2/MIH1
aaaatcaagaaacttcactgaatttggggcttcctccactatctttcgactctccactgcccgtaacggaaacgataccatccactaccgataacagcttgcatttgaaagctgatagcaacaaaaatcgcgatgcaagaactattgaaaatgatagtgaaattaagagtactaataatgctagtggctctggggcaaatcaatacacaactcttacttcaccttatcctatgaacgacattttgtacaacatgaacaatccgttacaatcaccgtcaccttcatcggtacctcaaaatccgactataaatcctcccataaatacagcaagtaacgaaactaatttatcgcctcaaacttcaaatggtaatgaaactcttatatctcctcgagcccaacaacatacgtccattaaagataatcgtctgtccttacctaatggtgctaattcgaatcttttcattgacactaacccaaacaatttgaacgaaaaactaagaaatcaattgaactcagatacaaattcatattctaactccatttctaattcaaactccaattctacgggtaatttaaattccagttattttaattcactgaacatagactccatgctagatgattacgtttctagtgatctcttattgaatgatgatgatgatgacactaatttatcacgccgaagatttagcgacgttataacaaaccaatttccgtcaatgacaaattcgaggaattctatttctcactctttggacctttggaaccatccgaaaattaatccaagcaatagaaatacaaatctcaatatcactactaattctacctcaagttccaatgcaagtccgaataccactactatgaacgcaaatgcagactcaaatattgctggcaacccgaaaaacaatgacgctaccatagacaatgagttgacacagattcttaacgaatataatatgaacttcaacgataatttgggcacatccacttctggcaagaacaaatctgcttgcccaagttcttttgatgccaatgctatgacaaagataaatccaagtcagcaattacagcaacagctaaaccgagttcaacacaagcagctcacctcgtcacataataacagtagcactaacatgaaatccttcaacagcgatctttattcaagaaggcaaagagcttctttacccataatcgatgattcactaagctacgacctggttaataagcaggatgaagaccccaagaacgatatgctgccgaattcaaatttgagttcatctcaacaatttatcaaaccgtctatgattctttcagacaatgcgtccgttattgcgaaagtggcgactacaggcttgagtaatgatatgccatttttgacagaggaaggtgaacaaaatgctaattctactccaaatttcgatctttccatcactcaaatgaatatggctccattatcgcctgcatcatcatcctccacgtctcttgcaacaaatcatttctatcaccatttcccacagcagggtcaccataccatgaactctaaaatcggttcttcccttcggaggcggaagtctgctgtgcctttgatgggtacggtgccgcttacaaatcaacaaaataatataagcagtagtagtgtcaactcaactggcaatggtgctggggttacgaaggaaagaaggccaagttacaggagaaaatcaatgacaccgtccagaagatcaagtgtcgtaatagaatcaacaaaggaactcgaggagaaaccgttccactgtcacatttgtcccaagagctttaagcgcagcgaacatttgaaaaggcatgtgagatctgttcactctaacgaacgaccatttgcttgtcacatatgcgataagaaatttagtagaagcgataatttgtcgcaacacatcaagactcataaaaaacatggagacatttaatagaccccatttttttaattcgatagatctttcttcataagataattctgttcaatgacttatgaagcttacggcttattgttattattatcatgattctttgccatttttccggtaccttcacattttgaaggaattcagttcccccgcctgtaacaacttagatacacgtagataaaaattaatatagccaaaatatttggtatgatagggtatagtataatataattaatacaatataatataatatagtacagtacagtataatattgtataatatcatatggcaccgtaatatcatatatatatccgtacagtttaatgaattccgtgcgcattatgctgtgcaatatgcctgtcagtttgaaaatcgaagataccacatcgtacaaaagcaagaggggtgcccctaatgggagaagattaccgtttttattttggggtagctaactgcgccacaaatagtatctaccagtttgctacttttagaactgtattatgtatctttgccgaatacttaatttcctaggcctcattttcaggactaaggatgaagaggggcttcaatgttttatatggcgctttttctgtccattgtatacgtcaaaaaagcatagatactccacttattagcggtaaagccctttttgatacattgttccggcgtttaaatattctcattatgatatttccccaacagttacactctgttatttccttcttttttatgctttatctgttttttccctgtttcgatgccgtatatcagaaaacttaaagttacaagcgaaaacaaagaaaagagagttaacttgaaaggagacaaaagataaacagagcagtggacaaaccaggattgaagtcagcgagggtgaagaaaccatgaacaatatatttcatggaactgaagatgaatgtgccaatgaagacgttcttagtttccaaaaaatttccttgaaaagtccctttggtaagaagaagaacatatttagaaatgttcagaccttctttaagtcaaaaagcaaacattcgaatgtcgacgatgatttaatcaataaagagaatcttgcctttgataaatctccattgttaacaaatcacaggagtaaggaaattgatggtccttcaccgaatataaagcagcttggccatcgcgatgagttagatgaaaatgaaaatgaaaatgatgatatagtcttaagcatgcattttgcttctcaaaccttacaaagtccaacaagaaactcatcaagaagatc

SEQ ID NO: 27 - DNA, 3460 bp, Artificial Sequence
  INO1-truncated VPS35/truncated SNA3
cgatctttcacgcagacatgcgactgcgcccgccgtagaccgtgacctggaagctcaccctgcagaggaatctcaagcacagcctccagcatatgatgaagacgatgaggccggtgccgatgtgcccttgatggacaacaaacaacagctctcttccggccgtacttagtgatcggaacgagctctttatcaccgtagttctaaataacacatagagtaaattattgcctttttcttcgttccttttgttcttcacgtcctttttatgaaatacgtgccggtgttccggggttggatgcggaatcgaaagtgttgaatgtgaaatatgcggaggccaagtatgcgcttcggcggctaaatgcggcatgtgaaaagtattgtctattttatcttcatccttctttcccagaatattgaacttatttaattcacatggagcagagaaagcgcacctctgcgttggcggcaatgttaatttgagacgtatataaattggagctttcgtcacctttttttggcttgttctgttgtcgggttcctaatgttagttttatccttgatttattctgtttcattcccttttttttccagtgaaaaagaagtaacaatgacagaagataatattgctccaatcacctccgttaaagtagttaccgacaagtgcacgtacaaggacaacgagctgctcaccaagtacagctacgaaaatgctgtagttacgaagacagctagtggccgcttcgatgtaacgcccactgttcaagactacgtgttcaaacttgacttgaaaaagccggaaaaactaggaattatgctcattgggttaggtggcaacaatggctccactttagtggcctcggtattggcgaataagcacaatgtggagtttcaaactaaggaaggcgttaagcaaccaaactacttcggctccatgactcaatgttctaccttgaaactgggtatcgatgcggaggggaatgacgtttatgctccttttaactctctgttgcccatggttagcccaaacgactttgtcgtctctggttgggacatcaataacgcagatctatacgaagctatgcagagaagtcaagttctcgaatatgatctgcaacaacgcttgaaggcgaagatgtccttggtgaagcctcttccttccatttactaccctgatttcattgcagctaatcaagatgagagagccaataactgcatcaatttggatgaaaaaggcaacgtaaccacgaggggtaagtggacccatctgcaacgcatcagacgcgatatccagaatttcaaagaagaaaacgcccttgataaagtaatcgttctttggactgcaaatactgagaggtacgtagaagtatctcctggtgttaatgacaccatggaaaacctcttgcagtctattaagaatgaccatgaagagattgctccttccacgatctttgcagcagcatctatcttggaaggtgtcccctatattaatggttcaccgcagaatacttttgttcccggcttggttcagctggctgagcatgagggtacattcattgcgggagacgatctcaagtcgggacaaaccaagttgaagtctgttctggcccagttcttagtggatgcaggtattaaaccggtctccattgcatcctataaccatttaggcaataatgacggttataacttatctgctccaaaacaatttaggtctaaggagatttccaaaagttctgtcatagatgacatcatcgcgtctaatgatatcttgtacaatgataaactgggtaaaaaagttgaccactgcattgtcatcaaatatatgaagcccgtcggggactcaaaagtggcaatggacgagtattacagtgagttgatgttaggtggccataaccggatttccattcacaatgtttgcgaagattctttactggctacgcccttgatcatcgatcttttagtcatgactgagttttgtacaagagtgtcctataagaaggtggacccagttaaagaagatgctggcaaattcgagaacttttatccagttttaaccttcttgagttactggttaaaagctccattaacaagaccaggatttcacccggtgaatggcttaaacaagcaaagaaccgccttagaaaattttttaagattgttgattggattgccttctcaaaacgaactaagattcgaagagagattgttgtaatctcatttcaacgactctctttttctttttccgcctacctataaaaaaacaagacattcaccattatcctattatcccttccatcaatacatatacttaacataacgtttataaaaattcaactatcaacagtctttatattttttttttttcttttgaactattgcctctttgtcacttcgtcttaaaggggcgttttttatttttttttttttttttcagttgaggtagatgcgagaaagtgctgtatttattcaagggccacctcagtaaagagaagaaaagagagaaaaaaaaaaagaaggtggtgtatgtgcgaccactcaacaaggcccagtgaagttaatatataacgataaaaggaggaggacgagaaagaagaagctgaaaaacacaatggcgtatgcggactcaccagaaaatgcgatcgctgttatcaagcagcgaaccgcactaatgaaccggtgtctatctcaacacaaactaatggaatcattacagcatacttccataatgttgaccgaattgagaaatccaaacttatcgccgaagaaatactacgaactttatgtcattattttcgactcattgactaatctatctacgtacctcatagaaaaccatcctcaaaatcaccacttagctgatctttatgagttggttcaatataccggtaacgtggtacccaggctttacttgatgatcacagttgggaccagctatctcactttcaatgaagcgcccaagaaggaaatcttaaaggatatgattgagatgtgtcgtggtgtgcaaaacccaataagaggtttgtttttacgctattatttatcccagagaaccaaagaattacttccggaggacgatccgtcgtttaactctcaatttattatgaataatttcatcgagatgaacaagttgtgggtgagattacaacaccaggggccacttcgtgagagggaaaccagaacacgtgaaagaaaagagctgcaaattttagtggggtctcaactagtacgtctttcgcagattattgatgataatttccaaatgtataagcaagatattcttcccaccattttggaacaagtcatacaatgtagagatttagtatcccaagaatatcttttggacgtcatctgccaagtgttcgcagacgagttccatttgaaaaccttggatactttactgcaaactactttgcatttgaaccctgatgtttcgataaacaagattgttctcactttggtcgat

SEQ ID NO: 28 - DNA, 2371 bp, Artificial Sequence
  DOG1-trucated YHR045W
tgattcgataggatcatcgcaaacaacaataagcttgtaccaatcctcgctgccgtttagcatcttaaaagacttccatgagtcgatgatcaggacatcaacatcttctagtcgttgtctaggaacagctggcaagaaatgaggtattgaacctgttcttatagaggtcatcatggaagccaaggagagagtaaaaccctctacggtagcaatagaattagctataccgatatttttgaaattcttattggaaagtttcggaaatatgcgcttggccataccattaagttcactcaacgaataacttttttctcttcctgtaaatttaattatgttttttcccttggaaacttccattatggcattccaaacatcaccgaaattgccatttcttattttgtattttaaagatagccctaatcctgtcgtcaatgggaatcctgtgggcactaaaatgcttcgataatatgctgtttcattttctttacgaacactggatatgctagattgctgtgacaaagccacccctgttaaatctctcttaaaatcaagtagaaaagagcttagcaaccagttggcgcccagcacaattaacaatattgttattaccagctgtagtagaaaagaccaattcattgctcatcaacttttgccgcttgtttaactgtctttctttctaacgaaagaactttctatagcttaattgaaaagcacttgaaatgcttaaaacttaacctatgttgaagctataaaggtgttgcagaatgatttccattcaatgtacttctcgaagttgctgtcgtttatgatacgatacatattagaactttttcattcttaacgacgcgcaaagaaaaacgaaaagctaatgaccaaaccaagggtcctaggattctcattaacatcaattcaaggcaatcagttacaatatttataataaagtaattcgtgctatgtatccttttgccaagtaacttgccagaaattccgtttatctacttcatcttatcggtgaaaaaatatacactgggctcttctacacacgccttctaccgcatatcctttagagtcttggcttcttgtaattttcaatcacggttatgttccgtgatacagacacgtcagagtattcctatatattccgacattaaccccgacatttcaaacaaccgttgttcagctatagaaggaagcgtatatctatgagattataggctctggtaccatctaatgatataaatttcatggaacttttttattgcaagtattttatttaattttattttcatttttaatgatcgcgattattctgttggaaataacgttctgatggagattgttggttacgttgccactcacgtaagaagttcaaaggataatggcagaattttcagctgatctatgtctttttgacctagatggtaccatagtgagtacaacagtggccgcagagaaagcatggaccaagttgtgttacgaatacggtgttgatccttccgagttatttaagcattctcatggtgcaagaacacaagaggttttgagaaggtttttccctaaattggatgatacagacaataaaggtgttcttgctctagaaaaagatattgcccatagttacttggacacagtaagccttattcctggtgcagagaacttactgttatcgttagatgtagatactgagactcaaaaaaagttacctgaaaggaaatgggctatcgttacctctggttctccatatttggcattttcatggttcgagacaatattgaaaaatgttggaaagcccaaagttttcattactgggtttgacgtgaagaacggtaagcctgatcccgagggttattcaagagctcgtgatttattgcgtcaagatttgcaattaactggtaaacaggatctgaagtatgttgtcttcgaagatgcacccgtgggcataaaggccggcaaagcaatgggcgccattactgtgggtataacatcctcgtatgacaagagcgttttatttgacgcaggagcagattatgtagtctgtgatttgacacaggtttccgtggttaagaacaatgaaaacggtattgtcatccaggtaaacaaccctttgacaagggcctgagtaaacaaaaatgtgacaaaagaacgaatatatatagatgtaaaacatatggacaagcaaaaagtcgaattatgtatgtcattttaggtactgaagaggtaagattttttttgagtttttcttcgaagatggttgtgtggttatatgttaatcttccttagcgcaaaacacttccatcaactgtatttcgttggaatgctttgtattcagttttgtatcattatctttaatcacaattgcgtcaggatgtaa

SEQ ID NO: 29 - DNA, 3061 bp, Artificial Sequence
  HAL1-trucated AIM45
gatcactatatttagttcgaaagttagtgtttgttttgccaagatatcctcgcagaaacagttttacttaaagtttcaggtgttgcgtggaatgcccctgctaagctgtcgtgtcgcgctcttccccgcgtttgtttcacattatatatcatatggcgtatgacggtatgggtgaaaataagcgtaggctggttgtgtgtatttctctcgcactttgaaagggaaaaataaaaatagagtctattaggaagctcacatatgccgggaaaaattacgtaaagcatcaaaagggaaagaaaatatagggaaagataaaacaaaaagcagaacggtatcaggcattcttgttttagcctttatgtacgatatggcgtttattatggagaatattgaggtattcaactatttaatacttgctataaattctgccaaactactaatcattccgtttatataactcaagaaaagagaaatacagaaacaaatagatcagatgcatttcaaagatttaggattgcatgactacactctcaaaaacttgatgtatgagaataattgctgtaaattttatgatgccgtggatgaaaacaacatctcatatgttttaaaatttgttccctcagatgtgacttcggaaggggatactttcccattcgtggatcgctttcaagtaaaggaaggtgtttttttggtatattcctcaaatgactttggaaaagaaggtacggactactttacttatactggtagtggtggaaatgaggttcacatctcgggcacctcttcagaagcaggaataaaaccgcagtttattgaaacttgccatccaaaacatcttaagcggggaacaaaagagcaggaagatataaatagtagtacctcaaagaaaagtgcagttatcaacaatttttcgggtgaaaaaacaccaaatccaaggccacagagttccaacatttcagaaagagagacgtatgtcggaatattgaacgtcaaatgtaaaaataagaactcatcgaaaatacgaagtgaaaaattggtaagctccgtcatcgaaacaaagcatacgccaggattggcatctattttatcgaaagaaggcactacatatccgaataatgcggacgggaaacatatcagtatcgtgaatccatcctcaaaaatatatcattcatcccataaacagattgttaaaacgcctatccctaagagtggcctttctccaattgagagatgccctttcaatggtcaaaatattaaatgctactcaccaagaccactagatcatgaaagtccccaacgtgatttcaataataactttcagctgagaatactgaagagctcggtgttgcaaaggagacaatcaacacagaatagttgaaaaattcgttggtactagcttcgggtcggttagctgcgctgctatgcatttgttaatatctccatcgaatttttgttgtttcgttcaataactttattacttcccccttggactctttcgttctatcgttctacaagtccagccaaaatttttcccctcttccttttcttttgttcacttcttagctcacttatataattatatactgatatttggattcttttgttgcaaatatgctctcccagatttttctgttgagatgattcatgctttacatggattgagcattagagagtaactatatccaatttcgtaagacgagtatctactttcccttgtccccagtaacctcaggaacgtgacaactacttttcttaaactgtcaacagccaatgataccgtattcaatgcatgtcttgggattaagccactttgattgagttccgtattagtagtgagaattaaatcttgcaagatataagaattactcaacagagacgagatactttcctttttttggttcttttgttgttacttggcttaatggacaaagtctgctcgcatattgtatatgttactcttacgatgtgactccgcccgtttattatgacttttcggtacatttttagggcctcgaacgaaaatctactaactaaaaattaaaaacaattaaaataatcaacaaacaaaatctagtgatataatttactaccattaacggtaaagcagctaattgttaatttctatgtttaaatcattggctgctgtcttgcctagagctagcaaggcaaagttcctccagaaaaattacgcctccactttagctttcattgaaagctcaaaagatggctctgtttcaaggtcatcattgagtttattggctgctgcacaaaagttgtctaaccctatcacagctgtaatcacaggtagcaaagctgaaaaaactgctgaggcgctaaaatcttcatattcatgcagcaatttagaaaagcttgtcatatttgaagattcaaaattagatacctgtcttcccgaacaactaactccgttattagtgaaactattaaaaggcggcgactattcacattttgttgtctcaaactcctctgttggaaaaagtgttttacctcgggtgggtgcgctcttggacgtccaacctgtttgtgaggttactgtaatcaaagatcctaagacctttataaggccaatttatgcaggtaacattatttctacaatagaatgccaggcagaaaaaaaactgttgattattagggcatcagcttttccaccaattgcagagggtagtatggattctgttaccattgagaagagaactgatattcctccttgtgacttaaatgttacctgggttaaaactattcttaccaagagtgaaaggcctgaacttacttctgcacagaacgtggtaactggtggaagggcactcaaggataaggagacatttgagaagctattatcgccgctagcagatgttttgcacgctgctataggtgccacaagagcttctgttgataatggactatgtgataattctctacaaatcggtcagactggtaaggtagtcgcaccaaatttgtatatagccattggcgtttctggtgcagttcagcatttagcgggaatgaaggattcgaaagttatcgttgccattaacaatgatcc

SEQ ID NO: 30 - DNA, 903 bp, Artificial Sequence
  TRP1 variant
caccttacgtacaatcttgatccggagcttttctttttttgccgattaagaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaag

SEQ ID NO: 31 - DNA, 1091 bp, Artificial Sequence
  trucated MRPL17 variant
gatctttactccaccctttatttgctcatagtacataaaactaatctacctctatatataatttctgttttttcctatgttccttttttttttttgttaaatggacatccttatgtattcacataactactgtgtttcaaatgtagcattatcctattctttattctctttttttaccgtattcctggttttttttcaaggtttagatgaaaatgaaaatgaaaaaaaaaaaaggaaagaaaacagttaaacaatcaataaactttatattctacgtaatcataagcgcttatccagtaccatgaaggtaaatttaatgttgaaaagagggcttgctactgcaactgcaactgccagttccgctccccccaagattaaagtcggagtactactgtcaagaatccctataattaaatcagaattaaatgaactagagaaaaaatactatgagtaccaatcagaactagaaaagagactaatgtggacgtttccggcatatttttatttcaaaaagggtactgtagcagaacacaaatttctatccctgcagaaaggacctatctccaaaaaaaatggcatttggtttcctagaggcataccggacattaaacatggcagagaaagaagtactaagcaagaagttaaactttctgatgacagtacagtagcatttagcaacaatcaaaaagagcaaagcaaagacgatgttaataggcccgtgattcccaacgacaggataacggaagcagataggtcaaatgatatgaagagccttgaaagacaattgagcaggaccttatatcttttggttaaggataaaagcggtacttggaaattccctaacttcgatctttctgatgaatctaagccgttacacgtacacgcagagaacgaattgaaattgttgagcggtgatcagatatacacttggtctgtttctgctacgcccataggtgttttgcaggacgagagaaataggactgctgagtttattgtgaagtcacacattttggctggaaaatttgatttggtggcgtcgaaaaatgatgcattcgaggattttgcttggctgacaaaaggtgagatcagtg